(19) United States
(12) Patent Application Publication
(10) Pub. No.: US 2024/0082166 A1
(43) Pub. Date: Mar. 14, 2024

(54) ORAL CAPSULE OF PARP INHIBITOR AND PREPARATION METHOD THEREOF

(71) Applicant: Impact Therapeutics (Shanghai), Inc, Shanghai (CN)

(72) Inventors: Sui Xiong CAI, Shanghai (CN); Ning MA, Jiangsu (CN); Liping ZHAO, Shanghai (CN); Chunhui LIU, Shanghai (CN); Zongfeng SHI, Shanghai (CN)

(21) Appl. No.: 18/552,434

(22) PCT Filed: Mar. 25, 2022
(86) PCT No.: PCT/CN2022/083127

§ 371 Date: Sep. 25, 2023

(30) Foreign Application Priority Data
Mar. 26, 2021 (CN) 202110327776.4

(51) Int. Cl. A61K 9/48; A61K 31/517; A61K 45/06; A61K 47/02; A61K 47/12; A61K 47/26; A61K 47/32; A61K 47/38

(52) U.S. Cl. CPC A61K 9/4866; A61K 31/517; A61K 45/06; A61K 47/02; A61K 47/12; A61K 47/26; A61K 47/32; A61K 47/38

(57) ABSTRACT

An oral capsule formulation of a PARP inhibitor and a preparation method. The oral capsule formulation comprises a solid dispersion powder of an active ingredient 5-fluoro-1-(4-fluoro-3-(4-(pyrimidin-2-yl)piperazine-1-carbonyl)benzyl)quinazoline-2,4(1H,3H)-dione, a filler, a disintegrant, a glidant, and a lubricant, wherein less than 10 wt. % of the active ingredient in the solid dispersion powder is in a crystalline form. The defects of the fluidity, hygroscopicity and cohesiveness of the solid dispersion powder which result in the difficulty of production scale up of capsule formulation are addressed, and thus commercial scale production can be achieved, and the prepared capsule exhibits proper dissolution rate, excellent storage stability, meanwhile with a reasonable production cost.

### TECHNICAL FIELD

The present disclosure relates to an oral capsule of PARP inhibitor and preparation method thereof.

### BACKGROUND

Poly(ADP-ribose) polymerase (PARP) catalyzes the addition of poly(ADP-ribose) to the target protein using NAD⁺ that is an important process in DNA repair. This is an essential process for maintaining DNA and chromosome integrity and stability, and for ensuring the survival of mammalian cells. PARP catalyzes the majority of the intracellular ADP-ribose polymerization reactions. Phase II clinical trial data have shown that PARP inhibitor Olaparib (AZD2281) is effective for the treatment of BRCA mutated breast cancer. Olaparib (Lynparza) was approved by EMEA and FDA for the treatment of BRCA-mutated ovarian cancer in December 2014. The applications of PARP inhibitors for the treatment of cancer are mainly based on two mechanisms. First, for cancer cells with DNA repair deficiency, such as BRCA1 or BRCA2 deficient triple-negative breast cancer cells and the like, PARP inhibitors can directly kill the cancer cells through the mechanism of synthetic lethality and function as anticancer drugs independently. According to statistics, about 10-15% of breast cancer patients have family history of genetic factors, in which the BRCA1 or BRCA2 gene mutations account for 15-20% of all hereditary breast cancers. Second, because of the rapid growth of cancer cells, DNA replication is much higher in cancer cells than in normal cells. Drugs that cause DNA damage will induce cancer cell death selectively. However, due to the presence of DNA repair enzymes such as PARP, the therapeutic effects of these drugs can not be fully materialized. By inhibiting the DNA repair mechanism, PARP inhibitors in combination with commonly used DNA-damaging chemotherapeutic anti-cancer drugs, such as temozolomide, can achieve synergy effects and greatly enhance the anticancer effects of DNA-damaging anticancer drugs. Furthermore, PARP inhibitors may also be used to treat diseases due to excessive cell death, including central nervous system diseases such as stroke and neurodegenerative diseases (Akinori Iwashita et al., 2004, J. Pharmacol. Exp. Thera., 310:425).

WO2012130166 discloses a compound 1-(arylmethyl)quinazoline-2,4(1H,3H)-dione as a PARP inhibitor and a synthesis method therefor, which comprises 5-fluoro-1-(4-fluoro-3-(4-(pyrimidin-2-yl)piperazine-1-carbonyl)benzyl)quinazoline-2,4(1H,3H)-dione and a synthesis method therefor.

WO2017167251 provides a preparation process for 1-(arylmethyl)quinazoline-2,4(1H,3H)-dione, which comprises a preparation method for 5-fluoro-1-(4-fluoro-3-(4-(pyrimidin-2-yl)piperazine-1-carbonyl)benzyl)quinazoline-2,4(1H,3H)-dione.

5-fluoro-1-(4-fluoro-3-(4-(pyrimidin-2-yl)piperazine-1-carbonyl)benzyl)quinazoline-2,4(1H,3H)-dione has a chemical structure shown as follows:

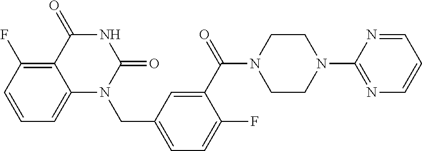 [SMILES: O=C(c1cc(Cn2c(=O)[nH]c(=O)c3c(F)cccc32)ccc1F)N1CCN(c2ncccn2)CC1]

WO2016155655 discloses a solid dispersion powder of 5-fluoro-1-(4-fluoro-3-(4-(pyrimidin-2-yl)piperazine-1-carbonyl)benzyl)quinazoline-2,4(1H,3H)-dione and a preparation method therefor, which comprises amorphous 5-fluoro-1-(4-fluoro-3-(4-(pyrimidin-2-yl)piperazine-1-carbonyl)benzyl)quinazoline-2,4(1H,3H)-dione and a polymer. Compared with crystallizing 5-fluoro-1-(4-fluoro-3-(4-(pyrimidin-2-yl)piperazine-1-carbonyl)benzyl)quinazoline-2,4(1H,3H)-dione, preparing the compound into an amorphous solid dispersion powder with smaller particle size increases the dissolution rate and the solubility of the compound and therefore improving its bioavailability.

The amorphous solid dispersion powder with a smaller particle size has poor fluidity, hygroscopicity and a certain cohesiveness. Filling the solid dispersion powder directly into capsule shells requires special filling equipment and scale-up production cannot be achieved. Furthermore, there is a tendency of aging and stability decline in storage for the solid dispersion formulation. As such, the shelf life of the solid dispersion formulations is generally shorter than that of a conventional formulation, which also greatly increases drug cost.

The solid dispersion powder of 5-fluoro-1-(4-fluoro-3-(4-(pyrimidin-2-yl)piperazine-1-carbonyl)benzyl)quinazoline-2,4(1H,3H)-dione used in the present disclosure has small particle size, fast dissolution rate, and high solubility and oral bioavailability. Although the solid dispersion powder can be directly filled in capsule shells for clinical use, the defects of poor fluidity, hygroscopicity, a certain cohesiveness of the solid dispersion powder which result in the impossibility in scale-up production cannot be addressed. In general, considering the defects in the physicochemical property of the solid dispersion of the compound, a process of direct mixing with excipients and capsule filling can be adopted; however, the problems of material delamination, poor mixing uniformity, and difficulty in smoothly filling capsules due to blockage of the filling equipment easily occur. The addition of a granulation step, if considered, is theoretically a possible way to mitigate the above problems, but it is also likely to have a negative effect on the dissolution and in vivo absorption of the drug, and also increase production cost.

Therefore, there is still a need of an applicable manufacturing process in the field that can solve the above problems and successfully achieve a commercial scale production of capsules, and the prepared capsules feature proper dissolution rate, excellent storage stability and meanwhile with a reasonable production cost.

### SUMMARY

In order to address the problem that capsule scale-up production cannot be achieved due to poor powder fluidity, hygroscopicity and a certain cohesiveness of the solid dispersion powder of 5-fluoro-1-(4-fluoro-3-(4-(pyrimidin-2-yl)piperazine-1-carbonyl)benzyl) quinazoline-2,4(1H,3H)-dione, the present disclosure provides a novel capsule formulation and a preparation method of direct mixing & capsule filling. By implementing the present disclosure, commercial scale production can be realized, and the prepared capsule features proper dissolution rate, excellent storage stability, and reasonable production cost.

In a first aspect, the present disclosure provides a pharmaceutical composition comprising a solid dispersion powder of an active ingredient 5-fluoro-1-(4-fluoro-3-(4-(pyrimidin-2-yl)piperazine-1-carbonyl)benzyl)quinazoline-2,4(1H,3H)-dione, a filler, a disintegrant, a glidant, and a lubricant, wherein less than 10 wt. %, preferably less than 5 wt. %, and more preferably less than 1 wt. % of the 5-fluoro-1-(4-fluoro-3-(4-(pyrimidin-2-yl)piperazine-1-carbonyl)benzyl)quinazoline-2,4(1H,3H)-dione in the solid dispersion powder is in a crystalline form.

In a second aspect, the present disclosure provides a pharmaceutical formulation, which is an oral capsule comprising the pharmaceutical composition according to any one of the embodiments of the present disclosure and a capsule shell; preferably, the capsule shell is selected from a plant capsule shell and a gelatin capsule shell, and more preferably, the capsule shell is the gelatin capsule shell.

In a third aspect, the present disclosure provides a method for preparing an oral capsule comprising a solid dispersion powder of 5-fluoro-1-(4-fluoro-3-(4-(pyrimidin-2-yl)piperazine-1-carbonyl)benzyl)quinazoline-2,4(1H,3H)-dione, wherein the method comprises:
(1) premixing the solid dispersion powder of an active ingredient 5-fluoro-1-(4-fluoro-3-(4-(pyrimidin-2-yl)piperazine-1-carbonyl)benzyl)quinazoline-2,4(1H,3H)-dione, a filler, a disintegrant, and a glidant to obtain a premixture;
(2) sieving the premixture obtained in the step (1) and then mixing again to obtain a first mixture;
(3) sieving a lubricant and adding the lubricant to the first mixture obtained in the step (2), and then mixing to obtain a final mixture; and
(4) filling the final mixture obtained in the step (3) into capsule shells to obtain the oral capsule.

In a fourth aspect, the present disclosure provides use of the pharmaceutical composition according to any one of the embodiments of the present disclosure in preparing a pharmaceutical formulation for the treatment or prevention of a PARP-mediated disease.

Detailed embodiments of various aspects of the present disclosure are described below.

### DETAILED DESCRIPTION

It should be understood that in the scope of the present disclosure, the above various technical features of the present disclosure and the technical features specifically described hereinafter (as in the examples) may be combined with each other to constitute a preferred technical scheme.

Unless otherwise defined, technical and scientific terms used herein have the same meaning as that commonly understood by those skilled in the art to which the present disclosure belongs.

As used herein, the terms “contain”, “comprise” or grammatical variations thereof mean that the compositions and methods described comprise the recited elements and do not exclude the others.

Unless expressly stated to the contrary, all ranges cited herein are inclusive; that is, the ranges include values for the upper and lower limits of the range and values therebetween. For example, temperature ranges, percentages, equivalent ranges and the like described herein include the upper and lower limits of the range and any value in the interval therebetween. Furthermore, it is to be understood that the sum of the weight percentages of all components in the pharmaceutical composition disclosed herein should equal 100%.

The compositions of the present disclosure comprise a mixture of an active ingredient with other chemical ingredients.

The “optional (optionally)” described herein represents that the object it modifies can be or cannot be selected. For example, the optional filler represents that the filler is or is not contained.

The present disclosure provides a pharmaceutical composition, and the pharmaceutical composition comprises a solid dispersion powder of an active ingredient 5-fluoro-1-(4-fluoro-3-(4-(pyrimidin-2-yl)piperazine-1-carbonyl)benzyl)quinazoline-2,4(1H,3H)-dione, a filler, a disintegrant, a glidant and a lubricant.

As used herein, the solid dispersion powder of 5-fluoro-1-(4-fluoro-3-(4-(pyrimidin-2-yl)piperazine-1-carbonyl)benzyl)quinazoline-2,4(1H,3H)-dione is preferably a solid dispersion as disclosed in PCT/CN2016/078262, which is incorporated herein by reference in its entirety. Preferably, the solid dispersion powder comprises an active ingredient 5-fluoro-1-(4-fluoro-3-(4-(pyrimidin-2-yl)piperazine-1-carbonyl)benzyl)quinazoline-2,4(1H,3H)-dione and a polymer hydroxypropyl methylcellulose phthalate. In the solid dispersion powder, the hydroxypropyl methylcellulose phthalate preferably accounts for 65-77% and more preferably 73-77% based on the total weight of the solid dispersion powder, and the active ingredient accounts for 25-33% based on the total weight of the solid dispersion powder. In a preferred embodiment, the solid dispersion powder consists of an active ingredient 5-fluoro-1-(4-fluoro-3-(4-(pyrimidin-2-yl)piperazine-1-carbonyl)benzyl)quinazoline-2,4(1H,3H)-dione and hydroxypropyl methylcellulose phthalate in a weight ratio of 1:2 to 1:3. In a particularly preferred embodiment, the solid dispersion powder consists of an active ingredient 5-fluoro-1-(4-fluoro-3-(4-(pyrimidin-2-yl)piperazine-1-carbonyl)benzyl)quinazoline-2,4(1H,3H)-dione and hydroxypropyl methylcellulose phthalate in a weight ratio of 1:2 to 1:3, and preferably, the solid dispersion powder consists of the active ingredient 5-fluoro-1-(4-fluoro-3-(4-(pyrimidin-2-yl)piperazine-1-carbonyl)benzyl)quinazoline-2,4(1H,3H)-dione and the hydroxypropyl methylcellulose phthalate in a weight ratio of 1:2 or 1:3.

In some other embodiments, the solid dispersion powder further comprises a surfactant. In some embodiments, the surfactant is poloxamer. Preferably, the surfactant has a content of 2-5% based on a total weight of the solid dispersion powder. In a preferred embodiment, the solid dispersion powder consists of 5-fluoro-1-(4-fluoro-3-(4-(pyrimidin-2-yl)piperazine-1-carbonyl)benzyl)quinazoline-2,4(1H,3H)-dione, hydroxypropyl methylcellulose phthalate and poloxamer in a weight ratio of 1:2.8:0.2.

Preferably, less than 10 wt. %, preferably less than 5 wt. %, more preferably less than 1 wt. %, and most preferably 0 wt. % of 5-fluoro-1-(4-fluoro-3-(4-(pyrimidin-2-yl)piperazine-1-carbonyl)benzyl)quinazoline-2,4(1H,3H)-dione in the solid dispersion powder used herein is in a crystalline form.

In the present application, the hydroxypropyl methylcellulose phthalate is hydroxypropyl methylcellulose phthalate that conforms to standards set forth in Chinese Pharmacopoeia. More specifically, the hydroxypropyl methylcellulose phthalate has a methoxy content of 12.0-28.0%, a 2-hydroxypropoxyl content of 4.0-23.0%, an acetyl content of 2.0-16.0%, and a succinoyl content of 4.0-28.0%, calculated on the dried basis.

In a preferred embodiment, the solid dispersion powder in the pharmaceutical composition has a content of 15-30%, preferably 15-22%, and more preferably 16-20% based on a total weight of the pharmaceutical composition.

In a preferred embodiment, the active ingredient 5-fluoro-1-(4-fluoro-3-(4-(pyrimidin-2-yl)piperazine-1-carbonyl)benzyl)quinazoline-2,4(1H,3H)-dione in the pharmaceutical composition has a content of 3.5-5.0%, preferably 4.0-5.0% based on a total weight of the pharmaceutical composition.

As used herein, the filler in the pharmaceutical composition may be selected from a group consisting of starch, sucrose, microcrystalline cellulose, anhydrous calcium hydrophosphate, mannitol, lactose, pregelatinized starch, glucose, maltose, cyclodextrin, cellulose, silicified microcrystalline cellulose and any combination thereof. The filler may have a content of 60-85%, preferably 70-82%, and more preferably 75-82% based on a total weight of the pharmaceutical composition.

Preferably, the filler comprises microcrystalline cellulose. Preferably, the microcrystalline cellulose has D90 of 170-480 μm. In some embodiments, D90 of the microcrystalline cellulose is 170-283 μm. In still other embodiments, D90 of the microcrystalline cellulose is 275-480 μm. The D90 was determined using a Malvern Mastersizer 2000 Laser Particle Size Analyzer (General Chapter 0982, Chinese Pharmacopoeia) with a refractive index of the test sample set as 1.45.

Preferably, the microcrystalline cellulose has a content of 10-60% based on a total weight of the pharmaceutical composition. In some embodiments, the microcrystalline cellulose has a content of 10-30%, preferably 15-28% based on a total weight of the pharmaceutical composition. In some embodiments, the microcrystalline cellulose has a content of 24-28% based on a total weight of the pharmaceutical composition. In some other embodiments, the microcrystalline cellulose has a content of 12-18% based on a total weight of the pharmaceutical composition. In some other embodiments, the microcrystalline cellulose has a content of 20-60%, preferably 25-60%, more preferably 35-60%, more preferably 38-55% based on a total weight of the pharmaceutical composition.

Preferably, the filler further comprises mannitol. Preferably, the particle size distribution of particles with a size of >75 μm of the mannitol is not less than 70%, preferably not less than 80%. In a particularly preferred embodiment, the inventors have found that the material fluidity and the capsule filling adaptability on automatic encapsulation equipment were further improved when the particle size distribution of particles with a size of >75 μm of the mannitol used is not less than 90%, with which the particle size of the filler is almost doubled compared to a situation that the particle size distribution of particles with a size of >75 μm of the mannitol used is not less than 70%. Meanwhile it has been surprisingly found that the increase in the particle size of excipients does not lead to the occurrence of delamination and uneven mixing of the materials during the mixing and capsule filling process. The scaled-up trial production results showed that, the uniformity of the mixing materials and the content uniformity of the finished capsule product are good, the defects of the physicochemical properties of the solid dispersion powder can be well overcome, and the required dissolution characteristic is achieved, meanwhile the requirements of the direct-mixing & capsule filling process are met. Thus, in a particularly preferred embodiment, the particle size distribution of the mannitol particles with a size of >75 μm is not less than 90%. The particle size distribution is determined using a laser particle sizer, wherein the vibration sampling speed is 15-30%, the Auger Speed is 30-45%, and the shading degree is 4-12%.

Preferably, the mannitol has a content of 25-70% based on a total weight of the pharmaceutical composition. In some embodiments, the mannitol has a content of 50-70%, preferably 50-68%, more preferably 50-65% based on a total weight of the pharmaceutical composition. In some embodiments, the mannitol has a content of 50-55% based on a total weight of the pharmaceutical composition. In some other embodiments, the mannitol has a content of 58-63%. In some embodiments, the mannitol has a content of 25-55% based on a total weight of the pharmaceutical composition. In some embodiments, the mannitol has a content of 25-45% based on a total weight of the pharmaceutical composition.

In a preferred embodiment, the filler in the pharmaceutical composition of the present disclosure is microcrystalline cellulose and mannitol, wherein D90 of the microcrystalline cellulose is 170-480 μm, preferably 275-480 μm, and the particle size distribution of particles with a size of >75 μm of the mannitol is not less than 70%, preferably not less than 80%, and more preferably not less than 90%.

In some embodiments, based on a total weight of the pharmaceutical composition, the microcrystalline cellulose has a content of 10-28%, preferably 15-28%, 24-28%, or 12-18%, and the mannitol has a content of 50-70%, 50-68%, 50-65%, 50-55% or 58-63%. In some other embodiments, based on a total weight of the pharmaceutical composition, the microcrystalline cellulose has a content of 25-55%, 35-55%, preferably 38-55%, and the mannitol has a content of 25-55%, preferably 25-43%. Preferably, based on a total weight of the pharmaceutical composition, a total weight of the microcrystalline cellulose and mannitol accounts for 60-85%, preferably 70-82%, more preferably 70-80%or 75-80% or 76-81%.

Preferably, in the pharmaceutical composition of the present disclosure, when the filler is mannitol and microcrystalline cellulose, the amount (by weight) of the mannitol is 0.5-7 times, such as 0.5-1 time, or 2-7 times the amount of the microcrystalline cellulose.

As used herein, the disintegrant in the pharmaceutical composition may be selected from sodium carboxymethyl starch, low-substituted hydroxypropyl cellulose, crospovidone, croscarmellose sodium, croscarmellose, methylcellulose, pregelatinized starch, sodium alginate, and any combination thereof. Preferably, the disintegrant is crospovidone, croscarmellose, or croscarmellose sodium. Generally, the disintegrant in the pharmaceutical composition may have a content of 0.1-10%, preferably 0.5-3% based on a total weight of the pharmaceutical composition. In a preferred embodiment, the disintegrant is crospovidone and/or croscarmellose sodium, and the crospovidone and/or the croscarmellose sodium have a content of 0.5-3% based on a total weight of the pharmaceutical composition. In some embodiments, the particle size (D90) of the crospovidone is controlled to be 270-385 μm.

As used herein, the glidant may be selected from powdered cellulose, magnesium trisilicate, colloidal silicon dioxide, talc, and any combination thereof. Preferably, the glidant is colloidal silicon dioxide. The glidant may have a content of 0.1-10%, preferably 0.5-3%, and more preferably 1-3% based on a total weight of the pharmaceutical composition.

As used herein, the lubricant may be selected from zinc stearate, glyceryl monostearate, glyceryl palmitostearate, magnesium stearate, sodium stearyl fumarate and any combination thereof. Preferably, the lubricant is magnesium stearate. The lubricant may have a content of 0.1-3%, preferably 0.3-1%, such as 0.5±0.1% based on a total weight of the pharmaceutical composition.

In some embodiments, the pharmaceutical composition described herein may also comprise a binder and/or a solubilizer.

It should be understood that various excipients contained in the pharmaceutical composition, such as a surfactant, a filler, a disintegrant, a glidant, a lubricant, a binder, and a solubilizer, are pharmaceutically acceptable excipients conventionally used in the art, and meet the requirements of pharmacopoeias of various countries.

In a particularly preferred embodiment, based on a total weight of the pharmaceutical composition, the pharmaceutical composition of the present application comprises:
16-20% of a solid dispersion powder, wherein the solid dispersion powder consists of an active ingredient 5-fluoro-1-(4-fluoro-3-(4-(pyrimidin-2-yl)piperazine-1-carbonyl)benzyl)quinazoline-2,4(1H,3H)-dione and hydroxypropyl methylcellulose phthalate in a weight ratio of 1:3, and less than 5 wt. % and preferably less than 1 wt. % of the 5-fluoro-1-(4-fluoro-3-(4-(pyrimidin-2-yl)piperazine-1-carbonyl)benzyl)quinazoline-2,4(1H,3H)-dione is in a crystalline form;
10-28% of microcrystalline cellulose, wherein D90 of the microcrystalline cellulose is 170-480 μm, preferably 275-480 μm;
50-70% of mannitol, wherein the particle size distribution of particles with a size of >75 μm of the mannitol is not less than 70%, preferably not less than 90%;
0.5-3% of crospovidone and/or croscarmellose sodium;
1-3% of colloidal silicon dioxide; and
0.3-1%, such as 0.5±0.2% or 0.5±0.1%, of magnesium stearate.

In some other particularly preferred embodiments, based on a total weight of the pharmaceutical composition, the pharmaceutical composition of the present application comprises:
16-20% of a solid dispersion powder, wherein the solid dispersion powder consists of an active ingredient 5-fluoro-1-(4-fluoro-3-(4-(pyrimidin-2-yl)piperazine-1-carbonyl)benzyl)quinazoline-2,4(1H,3H)-dione and hydroxypropyl methylcellulose phthalate in a weight ratio of 1:3, and less than 5 wt. % and preferably less than 1 wt. % of the 5-fluoro-1-(4-fluoro-3-(4-(pyrimidin-2-yl)piperazine-1-carbonyl)benzyl)quinazoline-2,4(1H,3H)-dione is in a crystalline form;
25-55% of microcrystalline cellulose, wherein D90 of the microcrystalline cellulose is 170-480 μm, preferably 275-480 μm;
25-55% of mannitol, wherein the particle size distribution of particles with a size of >75 μm of the mannitol is not less than 70%, preferably not less than 90%;
0.5-3% of crospovidone and/or croscarmellose sodium;
1-3% of colloidal silicon dioxide; and
0.3-1%, such as 0.5±0.2% or 0.5±0.1%, of magnesium stearate.

Preferably, for the pharmaceutical composition containing 15 mg or more of the 5-fluoro-1-(4-fluoro-3-(4-(pyrimidin-2-yl)piperazine-1-carbonyl)benzyl)quinazoline-2,4(1H,3H)-dione per dose, the content of microcrystalline cellulose may be in a range of 10-28% and the content of mannitol may be in a range of 50-68%, based on the total weight of the pharmaceutical composition. And, for the pharmaceutical composition containing less than 15 mg of the 5-fluoro-1-(4-fluoro-3-(4-(pyrimidin-2-yl)piperazine-1-carbonyl)benzyl) quinazoline-2,4(1H,3H)-dione per dose, the content of microcrystalline cellulose may be in a range of 25-55% and the content of mannitol may be in a range of 25-55%, based on the total weight of the pharmaceutical composition

In another aspect, the present disclosure provides a pharmaceutical formulation, which is an oral capsule comprising the pharmaceutical composition according to any one of the embodiments of the present disclosure and a capsule shell. Preferably, the capsule shell is selected from a plant capsule shell and a gelatin capsule shell, and more preferably, the capsule shell is a gelatin capsule shell. In a preferred embodiment, the capsule comprises 10-20 mg of the active ingredient 5-fluoro-1-(4-fluoro-3-(4-(pyrimidin-2-yl)piperazine-1-carbonyl)benzyl)quinazoline-2,4(1H,3H)-dione per capsule.

In a particularly preferred embodiment, the capsule is a capsule comprising 10 mg of the active ingredient per capsule, and the pharmaceutical composition in the capsule comprises:
16-20% of a solid dispersion powder, wherein the solid dispersion powder consists of an active ingredient 5-fluoro-1-(4-fluoro-3-(4-(pyrimidin-2-yl)piperazine-1-carbonyl) benzyl)quinazoline-2,4(1H,3H)-dione and hydroxypropyl methylcellulose phthalate in a weight ratio of 1:3, and less than 5 wt. % and preferably less than 1 wt. % of the 5-fluoro-1-(4-fluoro-3-(4-(pyrimidin-2-yl)piperazine-1-carbonyl)benzyl)quinazoline-2,4(1H,3H)-dione is in a crystalline form;
23-28% of microcrystalline cellulose, wherein D90 of the microcrystalline cellulose is 170-480 μm, preferably 275-480 μm;
50-55% of mannitol, wherein the particle size distribution of particles with a size of >75 μm of the mannitol is not less than 70%, preferably not less than 80%, and more preferably not less than 90%;
0.5-3% of crospovidone and/or croscarmellose sodium;
1-3% of colloidal silicon dioxide; and
0.3-1%, such as 0.5±0.1%, of magnesium stearate.

In another particularly preferred embodiment, the capsule is a capsule comprising 20 mg of the active ingredient per capsule, and the pharmaceutical composition in the capsule comprises:
16-20% of a solid dispersion powder, wherein the solid dispersion powder consists of an active ingredient 5-fluoro-1-(4-fluoro-3-(4-(pyrimidin-2-yl)piperazine-1-carbonyl) benzyl)quinazoline-2,4(1H,3H)-dione and hydroxypropyl methylcellulose phthalate in a weight ratio of 1:3, and less than 5 wt. % and preferably less than 1 wt. % of the 5-fluoro-1-(4-fluoro-3-(4-(pyrimidin-2-yl)piperazine-1-carbonyl)benzyl)quinazoline-2,4(1H,3H)-dione is in a crystalline form;
10-28% of microcrystalline cellulose, wherein D90 of the microcrystalline cellulose is 170-480 μm. preferably 275-480 μm;
50-70% of mannitol, wherein a particle size distribution of particles with a size of >75 μm of the mannitol is not less than 70%, preferably not less than 90%;
0.5-3% of crospovidone and/or croscarmellose sodium;
1-3% of colloidal silicon dioxide; and

10.3-1%, such as 0.5±0.1%, of magnesium stearate.

Intermediates of the capsule of the present disclosure have good fluidity and are applicable for capsule filling after being directly mixed. No granulation is required, which simplifies the whole process steps and reduces the impact of the formulation process on product bioavailability. The compositions for capsule formulation described above can result in a drug product featuring satisfactory stability, dissolution property that meets the bioavailability requirement, and reasonable production cost.

In another aspect, the present disclosure provides a method for preparing an oral capsule comprising a solid dispersion powder of 5-fluoro-1-(4-fluoro-3-(4-(pyrimidin-2-yl)piperazine-1-carbonyl)benzyl)quinazoline-2,4(1H,3H)-dione, wherein the method comprises:
(1) premixing the solid dispersion powder of an active ingredient 5-fluoro-1-(4-fluoro-3-(4-(pyrimidin-2-yl)piperazine-1-carbonyl)benzyl)quinazoline-2,4(1H,3H)-dione, a filler, a disintegrant, and a glidant to obtain a premixture;
(2) sieving the premixture obtained in the step (1) and then mixing again to obtain a first mixture;
(3) sieving a lubricant and adding the lubricant to the first mixture obtained in the step (2), and then mixing to obtain a final mixture; and
(4) filling the final mixture obtained in the step (3) into a capsule shell to obtain the oral capsule.

Preferably, the premixing in the step (1) is performed at a rotation speed of 3-40 rpm for 2-20 min. In some embodiments, the premixing in the step (1) is performed at a rotation speed of 3-20 rpm, preferably 3-8 rpm, for 2-8 min, preferably 3-5 min.

Preferably, the sieving in the step (2) is performed by using a vacuum negative pressure sieve, and a size of a screen used for sieving is 20-40 meshes, preferably 30 meshes.

Preferably, the first mixture in the step (2) is obtained by mixing the premixture at a rotation speed of 3-40 rpm for 3-20 min. In some embodiments, the first mixture in the step (2) is obtained by mixing the premixture at a rotation speed of 3-20 rpm, preferably 3-8 rpm, for 3-15 min, preferably 6-10 min.

Preferably, a size of a screen used for sieving in the step (3) is 20-40 meshes, preferably 30 meshes.

Preferably, the mixing in the step (3) is performed at a rotation speed of 3-40 rpm for 2-20 min. In some embodiments, the mixing in the step (3) is performed at a rotation speed of 3-20 rpm, preferably 3-8 rpm, for 2-20 min, preferably 6-10 min.

In the method for preparing the oral capsule, when the mixing in the steps (1) to (3) is insufficient, the solid dispersion powder of 5-fluoro-1-(4-fluoro-3-(4-(pyrimidin-2-yl)piperazine-1-carbonyl)benzyl)quinazoline-2,4(1H,3H)-dione will be unevenly distributed in the mixed powder; when the mixing in the steps (1) to (3) is excessive, the delamination and separation of the solid dispersion powder of 5-fluoro-1-(4-fluoro-3-(4-(pyrimidin-2-yl)piperazine-1-carbonyl)benzyl)quinazoline-2,4(1H,3H)-dione and the excipients will occur, which will affect product quality.

Preferably, the method for preparing an oral capsule comprising a solid dispersion powder of 5-fluoro-1-(4-fluoro-3-(4-(pyrimidin-2-yl)piperazine-1-carbonyl)benzyl) quinazoline-2,4(1H,3H)-dione described herein comprises:
(1) premixing the solid dispersion powder of the active ingredient 5-fluoro-1-(4-fluoro-3-(4-(pyrimidin-2-yl)piperazine-1-carbonyl)benzyl)quinazoline-2,4(1H,3H)-dione, the filler, the disintegrant, and the glidant at a rotation speed of 6 rpm for 3 min to obtain a premixture;
(2) performing manual sieving and mixing the sieved premixture at a rotation speed of 6 rpm for 10 min to obtain a first mixture, wherein a size of a screen used for sieving is 30 meshes;
(3) sieving the lubricant with a 30-mesh sieve, adding the sieved lubricant into the first mixture in the step (2), and mixing at a rotation speed of 6 rpm for 15 min to obtain a final mixture; and
(4) filling the final mixture obtained in the step (3) into capsule shells to obtain the oral capsule.

Preferably, the method for preparing an oral capsule comprising a solid dispersion powder of 5-fluoro-1-(4-fluoro-3-(4-(pyrimidin-2-yl)piperazine-1-carbonyl)benzyl) quinazoline-2,4(1H,3H)-dione described herein comprises:
(1) premixing the solid dispersion powder of the active ingredient 5-fluoro-1-(4-fluoro-3-(4-(pyrimidin-2-yl)piperazine-1-carbonyl)benzyl)quinazoline-2,4(1H,3H)-dione, the filler, the disintegrant, and the glidant at a rotation speed of 6 rpm for 3 min to obtain a premixture;
(2) sieving by using a vacuum negative pressure sieve and mixing the sieved premixture at a rotation speed of 6 rpm for 10 min to obtain a first mixture, wherein a size of a screen used for sieving is 30 meshes;
(3) sieving the lubricant with a 30-mesh sieve, adding the sieved lubricant into the first mixture in the step (2), and mixing at a rotation speed of 6 rpm for 3 min to obtain a final mixture; and
(4) filling the final mixture obtained in the step (3) into capsule shells to obtain the oral capsule.

The capsule preparation method described above relates to the steps of direct mixing and capsule filling, thus the method is a granulation-free process, which can simplify the whole process steps and reduce the impact of the formulation process on product bioavailability, and the drug crystalline form (amorphous state) of the solid dispersion powder of 5-fluoro-1-(4-fluoro-3-(4-(pyrimidin-2-yl)piperazine-1-carbonyl)benzyl) quinazoline-2,4(1H,3H)-dione is not changed in the process.

According to the method described above, the solid dispersion powder of 5-fluoro-1-(4-fluoro-3-(4-(pyrimidin-2-yl)piperazine-1-carbonyl)benzyl)quinazoline-2,4(1H,3H)-dione is premixed with excipients, so that the problems that the solid dispersion powder has poor fluidity, is easy to agglomerate during storage, and is difficult to be sieved alone are effectively solved, and meanwhile, sieving after premixing can pulverize the agglomerates of the solid dispersion powder, and finally ensure the uniformity of the drug content. Furthermore, uniformly mixing the solid dispersion powder and the excipients in steps can improve content uniformity of the product. Meanwhile, only reasonable process parameters, such as a mixing condition with non-excessive lubricant, can ensure the dissolution rate of the product.

In another aspect, the present disclosure also provides therapeutic use of the pharmaceutical composition according to any one of the embodiments of the present disclosure in preparing a pharmaceutical formulation for the treatment or prevention of a PARP-mediated disease. Preferably, the pharmaceutical formulation is an oral capsule. Also provided is a pharmaceutical composition according to any one of the embodiments of the present disclosure for use in the treatment or prevention of a PARP-mediated disease.

Preferably, the PARP-mediated disease is cancer. Exemplary cancers include solid tumors and blood tumors, such as liver cancer, melanoma, Hodgkin's disease, non-Hodgkin's lymphoma, acute lymphatic leukemia, chronic lymphatic leukemia, multiple myeloma, neuroblastoma, breast cancer, ovarian cancer, lung cancer, Wilms' tumor, cervical cancer, testicular cancer, soft tissue sarcoma, chronic lymphocytic leukemia, primary macroglobulinemia, bladder cancer, chronic myelogenous leukemia, primary brain cancer, malignant melanoma, small cell lung cancer, stomach cancer, colon cancer, malignant pancreatic insulinoma, malignant carcinoid cancer, choriocarcinoma, mycosis fungoides, head and neck cancer, osteoganic sarcoma, pancreatic cancer, acute myeloid leukemia, hairy cell leukemia, rhabdomyosarcoma, Kaposi's sarcoma, genitourinary tumor, thyroid cancer, esophageal cancer, malignant hypercalcemia, cervical hyperplasia, renal cell carcinoma, endometrial cancer, polycythemia vera, essential thrombocythemia, adrenocortical carcinoma, skin cancer and prostate cancer.

In some embodiments, the pharmaceutical formulation is a combination of drugs and comprises the pharmaceutical composition according to any one of the embodiments of the present disclosure and at least one known anti-cancer drug or a pharmaceutically acceptable salt of the anti-cancer drug. The pharmaceutical composition and the at least one known anti-cancer drug or the pharmaceutically acceptable salt thereof may be prepared in the form of an independent pharmaceutical product or in the form of a mixture of both. Preferably, the known anti-cancer drug can be selected from one or more of the following anti-cancer drugs: busulfan, melphalan, chlorambucil, cyclophosphamide, ifosfamide, temozolomide, bendamustine, cisplatin, mitomycin C, bleomycin, carboplatin, camptothecin, irinotecan, topotecan, doxorubicin, epirubicin, aclacinomycin, mitoxantrone, methylhydroxyellipticine, etoposide, 5-azacytidine, gemcitabine, 5-fluorouracil, methotrexate, 5-fluoro-2′-deoxyuridine, fludarabine, nelarabine, cytarabine, alanosine, pralatrexate, pemetrexed, hydroxyurea, thioguanine, colchicine, vinblastine, vincristine, vinorelbine, paclitaxel, ixabepilone, cabazitaxel, docetaxel, alemtuzumab (Campath), panitumumab, ofatumumab, bevacizumab, herceptin, rituximab, imatinib, gefitinib, erlotinib, lapatinib, sorafenib, sunitinib, nilotinib, dasatinib, pazopanib, temsirolimus, everolimus, vorinostat, romidepsin, tamoxifen, letrozole, fulvestrant, mitoguazone, octreotide, retinoic acid, arsenic trioxide, zoledronic acid, bortezomib, thalidomide or lenalidomide.

In some embodiments, the pharmaceutical composition or pharmaceutical formulation according to any one of the embodiments of the present disclosure may be used in combination with a radiation therapy.

Also provided herein is a method for treating or preventing a PARP-mediated disease, which comprises administering to a subject in need a therapeutically or prophylactically effective amount of the pharmaceutical composition or pharmaceutical formulation according to any one of the embodiments of the present disclosure.

As used herein, “prevention”, “prevent” and “preventing” include reducing the likelihood of the occurrence or exacerbation of a disease or disorder in a patient; the term also includes: preventing the occurrence of a disease or disorder in a mammal, particularly when such a mammal is predisposed to the disease or disorder but has not yet been diagnosed as having it. “Treatment” and other similar synonyms include the following meanings: (i) inhibiting a disease or disorder, i.e., arresting its development; (ii) alleviating the disease or disorder, i.e., causing regression of the disease or disorder; or (iii) alleviating symptoms caused by the disease or disorder.

As used herein, “administering” refers to a method capable of delivering a compound or composition to a desired site for a biological action. Administration methods well known in the art may be used in the present disclosure. As used herein, the preferred administration route is oral administration.

As used herein, the therapeutically and prophylactically effective amounts refer to amounts of the pharmaceutical compositions or pharmaceutical formulations of the present application that, when administered to a subject, are effective to prevent or ameliorate one or more symptoms of a disease or condition or the progression of the disease or condition. The specific effective amount will depend upon various factors, such as a particular disease to be treated, the physical conditions of a patient, e.g., weight, age and sex, the duration of the treatment, the co-administered treatment (if any), and the specific formulation composition used.

In some embodiments, the treatment or prevention method further comprises simultaneously or sequentially administering to a subject in need at least one known anti-cancer drug described herein or a pharmaceutically acceptable salt thereof, and/or a radiation therapy.

The following examples may help those skilled in the art more comprehensively understand the present disclosure, but are not intended to limit the present disclosure in any way. All the excipients can be commercially available.

### EXAMPLE 1

### Preparation of Solid Dispersion Powder of 5-fluoro-1-(4-fluoro-3-(4-(pyrimidin-2-yl)piperazine-1-carbonyl)benzyl)quinazoline-2,4(1H,3H)-dione

The formulation of the solid dispersion is shown below:

| | Weight |
|---|---|
| Component | percentage |
| 5-fluoro-1-(4-fluoro-3-(4-(pyrimidin-2-yl)piperazine- | 25.0% |
| 1-carbonyl)benzyl)quinazoline-2,4(1H,3H)-dione | |
| Hydroxypropyl methylcellulose phthalate HP-55 | 75.0% |

Preparation Method:

5-fluoro-1-(4-fluoro-3-(4-(pyrimidin-2-yl)piperazine-1-carbonyl)benzyl)quinazoline-2,4(1H,3H)-dione and hydroxypropyl methylcellulose phthalate were dissolved in a mixed solution of tetrahydrofuran and methanol (7:3, v/v), then spray drying was performed using a spray dryer, and the collected spray-dried dispersion was dried in a vacuum dryer to obtain a solid dispersion powder of 5-fluoro-1-(4-fluoro-3-(4-(pyrimidin-2-yl)piperazine-1-carbonyl)benzyl)quinazoline-2,4(1H,3H)-dione. According to detection, less than 1% of 5-fluoro-1-(4-fluoro-3-(4-(pyrimidin-2-yl)piperazine-1-carbonyl)benzyl)quinazoline-2,4(1H,3H)-dione was in a crystalline form in the solid dispersion powder.

### EXAMPLE 2

### Preparation of Oral Capsule Comprising Solid Dispersion Powder of 5-fluoro-1-(4-fluoro-3-(4-(pyrimidin-2-yl)piperazine-1-carbonyl)benzyl)quinazoline-2,4(1H,3H)-dione

The formulation of capsule comprising 10 mg of 5-fluoro-1-(4-fluoro-3-(4-(pyrimidin-2-yl)piperazine-1-carbonyl)benzyl)quinazoline-2,4(1H,3H)-dione per capsule is shown below:

| | Weight |
|---|---|
| Component | (mg) |
| Solid dispersion powder of | 40.00 |
| 5-fluoro-1-(4-fluoro-3-(4-(pyrimidin-2-yl)piperazine- | |
| 1-carbonyl)benzyl)quinazoline-2,4(1H,3H)-dione | |
| Microcrystalline cellulose PH102* | 59.80 |
| Mannitol 100SD** | 119.85 |
| Croscarmellose sodium | 6.90 |
| Colloidal silicon dioxide | 2.30 |
| Magnesium stearate | 1.15 |
| Empty gelatin capsules 0# | 1 capsule |
| Total amount | 230.00 |

*The particle size (D90) of the microcrystalline cellulose was controlled to be 170-283 μm.
**The particle size distribution of particles with a size of >75 μm (200 meshes) of the mannitol was controlled to be not less than 70%.

Preparation Method:

The solid dispersion powder of 5-fluoro-1-(4-fluoro-3-(4-(pyrimidin-2-yl)piperazine-1-carbonyl)benzyl)quinazoline-2,4(1H,3H)-dione prepared in Example 1, microcrystalline cellulose, mannitol, croscarmellose sodium, and colloidal silicon dioxide were added to a universal mixer and mixed at a rotation speed of 40 rpm for 5 min. Then sieving was performed by using a 40-mesh screen, and the sieved material was mixed at a rotation speed of 40 rpm for 8 min. Magnesium stearate was sieved through a 30-mesh sieve and added to a universal mixer, and then mixing was performed at a rotation speed of 40 rpm for 3 min. The obtained mixed powders were filled into empty gelatin capsule shells to obtain oral capsules.

### EXAMPLE 3

### PK Study in Dogs

The capsules prepared in Example 2 and the direct-filling capsules of the solid dispersion powder of 5-fluoro-1-(4-fluoro-3-(4-(pyrimidin-2-yl)piperazine-1-carbonyl)benzyl)quinazoline-2,4(1H,3H)-dione (the formulation and preparation method of the solid dispersion powder were as described in Example 1) were subjected to in vivo double crossover PK experimental study in dogs. In phase I, the first group of experimental dogs were orally administered with the capsule in Example 2, and the second group was administered with the solid dispersion powder direct-filling capsule; after a 7-day withdrawal period, phase II was conducted, in which the first group was administered with the solid dispersion powder direct-filling capsule, and the second group was administered with the capsule in Example 2. Each group had 3 male beagle dogs; all experimental dogs were fasted and fed 4 hours after administration. The administration dose was 0.8 mg/kg. The experiment results are shown in Table 1. According to T test, Cₘₐₓ, Tₘₐₓ and AUC₀₋ₗₐₛₜ of the capsule prepared in Example 2 and the solid dispersion powder direct-filling capsule in dogs have no significant difference (p>0.05), which shows that the capsule formulation composition and preparation process in Example 2 has no obvious impact on the in vivo absorption of the drug.

TABLE 1
Results of double crossover PK test

| | | Solid dispersion | |
|---|---|---|---|
| | Capsules in | powder direct- | |
| | Example 2, | filling capsule, | |
| PK parameters | average value | average value | P value |
| Cₘₐₓ (ng/ml) | 211 ± 197 | 236 ± 129 | 0.800 |
| Tₘₐₓ (h) | 1.19 ± 0.702 | 0.889 ± 0.612 | 0.440 |
| T_{1/2} (h) | 7.06 ± 6.22 | 3.04 ± 1.76 | — |
| Tₗₐₛₜ (h) | 19.3 ± 7.23 | 14.7 ± 7.23 | — |
| AUC₀₋ₗₐₛₜ (ng · h/ml) | 603 ± 361 | 723 ± 323 | 0.557 |
| AUC_{0-inf} (ng · h/ml) | 734 ± 497 | 750 ± 334 | — |
| MRT₀₋ₗₐₛₜ (h) | 4.25 ± 2.15 | 3.48 ± 0.983 | — |
| MRT_{0-inf} (h) | 8.35 ± 10.3 | 4.14 ± 1.53 | — |
| AUC_{Extra} (%) | 10.9 ± 18.5 | 3.84 ± 2.29 | — |
| AUMC^{_{Extra}} (%) | 30.0 ± 27.8 | 16.7 ± 10.1 | — |

### EXAMPLE 4

### Preparation of Oral Capsule Comprising Solid Dispersion Powder of 5-fluoro-1-(4-fluoro-3-(4-(pyrimidin-2-yl)piperazine-1-carbonyl)benzyl)quinazoline-2,4(1H,3H)-dione

The formulation of the capsule comprising 10 mg of 5-fluoro-1-(4-fluoro-3-(4-(pyrimidin-2-yl)piperazine-1-carbonyl)benzyl)quinazoline-2,4(1H,3H)-dione per capsule is shown below:

| Component | Weight (mg) |
|---|---|
| Solid dispersion powder of | 40.00 |
| 5-fluoro-1-(4-fluoro-3-(4-(pyrimidin-2-yl)piperazine- | |
| 1-carbonyl)benzyl)quinazoline-2,4(1H,3H)-dione | |
| Microcrystalline cellulose PH102* | 60.95 |
| Mannitol 100SD** | 121.00 |
| Croscarmellose sodium | 4.60 |
| Colloidal silicon dioxide | 2.30 |
| Magnesium stearate | 1.15 |
| Empty gelatin capsules 0# | 1 capsule |
| Total amount | 230.00 |

*The particle size (D90) of the microcrystalline cellulose was controlled to be 170-283 μm.
**The particle size distribution of particles with a size of >75 μm (200 meshes) of the mannitol was controlled to be not less than 70%.

Preparation Method:

The solid dispersion powder of 5-fluoro-1-(4-fluoro-3-(4-(pyrimidin-2-yl)piperazine-1-carbonyl)benzyl)quinazoline-2,4(1H,3H)-dione prepared in Example 1, microcrystalline cellulose, mannitol, croscarmellose sodium, and colloidal silicon dioxide were added to a mixer and mixed at a rotation speed of 15 rpm for 3 min. Then sieving was performed by using a 30-mesh screen, and the sieved material was mixed at a rotation speed of 15 rpm for 10 min. Magnesium stearate was sieved through a 30-mesh sieve and added to the mixer, and then mixing was performed at a rotation speed of 15 rpm for 3 min. The obtained mixed powders were filled into empty gelatin capsule shells to obtain oral capsules.

### EXAMPLE 5

### Preparation of Oral Capsule Comprising Solid Dispersion Powder of 5-fluoro-1-(4-fluoro-3-(4-(pyrimidin-2-yl)piperazine-1-carbonyl)benzyl)quinazoline-2,4(1H,3H)-dione

The formulation of capsule comprising 10 mg of 5-fluoro-1-(4-fluoro-3-(4-(pyrimidin-2-yl)piperazine-1-carbonyl)benzyl)quinazoline-2,4(1H,3H)-dione per capsule is shown below:

| Component | Weight (mg) |
|---|---|
| Solid dispersion powder of | 40.00 |
| 5-fluoro-1-(4-fluoro-3-(4-(pyrimidin-2-yl)piperazine- | |
| 1-carbonyl)benzyl)quinazoline-2,4(1H,3H)-dione | |
| Microcrystalline cellulose PH102* | 62.10 |
| Mannitol 100SD** | 122.15 |
| Croscarmellose sodium | 2.30 |
| Colloidal silicon dioxide | 2.30 |
| Magnesium stearate | 1.15 |
| Empty gelatin capsules 0# | 1 capsule |
| Total amount | 230.00 |

*The particle size (D90) of the microcrystalline cellulose was controlled to be 170-283 μm.
**The particle size distribution of particles with a size of >75 μm (200 meshes) of the mannitol was controlled to be not less than 70%.

Preparation Method:

The solid dispersion powder of 5-fluoro-1-(4-fluoro-3-(4-(pyrimidin-2-yl)piperazine-1-carbonyl)benzyl)quinazoline-2,4(1H,3H)-dione prepared in Example 1, microcrystalline cellulose, mannitol, croscarmellose sodium, and colloidal silicon dioxide were added to a mixer and mixed at a rotation speed of 15 rpm for 3 min. Then sieving was performed by using a 30-mesh screen, and the sieved material was mixed at a rotation speed of 15 rpm for 10 min. Magnesium stearate was sieved through a 30-mesh sieve and added to the mixer, and then mixing was performed at a rotation speed of 15 rpm for 3 min. The obtained mixed powders were filled into empty gelatin capsule shells to obtain oral capsules.

### EXAMPLE 6

### Preparation of Oral Capsule Comprising Solid Dispersion Powder of 5-fluoro-1-(4-fluoro-3-(4-(pyrimidin-2-yl)piperazine-1-carbonyl)benzyl)quinazoline-2,4(1H,3H)-dione

The formulation of capsule comprising 10 mg of 5-fluoro-1-(4-fluoro-3-(4-(pyrimidin-2-yl)piperazine-1-carbonyl)benzyl)quinazoline-2,4(1H,3H)-dione per capsule is shown below:

| | Weight |
|---|---|
| Component | (mg) |
| Solid dispersion powder of | 40.00 |
| 5-fluoro-1-(4-fluoro-3-(4-(pyrimidin-2-yl)piperazine- | |
| 1-carbonyl)benzyl)quinazoline-2,4(1H,3H)-dione | |
| Microcrystalline cellulose PH102* | 61.80 |
| Mannitol 100SD** | 123.60 |
| Croscarmellose sodium | 1.15 |
| Colloidal silicon dioxide | 2.30 |
| Magnesium stearate | 1.15 |
| Empty gelatin capsules 0# | 1 capsule |
| Total amount | 230.00 |

*The particle size (D90) of the microcrystalline cellulose was controlled to be 170-283 μm.
**The particle size distribution of particles with a size of >75 μm (200 meshes) of the mannitol should be controlled to be not less than 70%.

Preparation Method:

The solid dispersion powder of 5-fluoro-1-(4-fluoro-3-(4-(pyrimidin-2-yl)piperazine-1-carbonyl)benzyl)quinazoline-2,4(1H,3H)-dione prepared in Example 1, microcrystalline cellulose, mannitol, croscarmellose sodium, and colloidal silicon dioxide were added to a mixer and mixed at a rotation speed of 15 rpm for 3 min. Then sieving was performed by using a 30-mesh screen, and the sieved material was mixed at a rotation speed of 15 rpm for 10 min. Magnesium stearate was sieved through a 30-mesh sieve and added to the mixer, and then mixing was performed at a rotation speed of 15 rpm for 3 min. The obtained mixed powders were filled into empty gelatin capsule shells to obtain oral capsules.

### EXAMPLE 7

### Preparation of Oral Capsule Comprising Solid Dispersion Powder of 5-fluoro-1-(4-fluoro-3-(4-(pyrimidin-2-yl)piperazine-1-carbonyl)benzyl)quinazoline-2,4(1H,3H)-dione

The formulation of capsule comprising 10 mg of 5-fluoro-1-(4-fluoro-3-(4-(pyrimidin-2-yl)piperazine-1-carbonyl)benzyl)quinazoline-2,4(1H,3H)-dione per capsule is shown below:

| Component | Weight (mg) |
|---|---|
| Solid dispersion powder of | 40.00 |
| 5-fluoro-1-(4-fluoro-3-(4-(pyrimidin-2-yl)piperazine- | |
| 1-carbonyl)benzyl)quinazoline-2,4(1H,3H)-dione | |
| Microcrystalline cellulose PH102* | 61.80 |
| Mannitol 100SD** | 123.60 |
| Crospovidone XL*** | 1.15 |
| Colloidal silicon dioxide | 2.30 |
| Magnesium stearate | 1.15 |
| Empty gelatin capsules 0# | 1 capsule |
| Total amount | 230.00 |

*The particle size (D90) of the microcrystalline cellulose was controlled to be 170-283 μm.
**The particle size distribution of particles with a size of >75 μm (200 meshes) of the mannitol should be controlled to be not less than 70%.
***The particle size (D90) of the crospovidone is controlled to be 270-385 μm.

Preparation Method:

The solid dispersion powder of 5-fluoro-1-(4-fluoro-3-(4-(pyrimidin-2-yl)piperazine-1-carbonyl)benzyl)quinazoline-2,4(1H,3H)-dione prepared in Example 1, microcrystalline cellulose, mannitol, crospovidone, and colloidal silicon dioxide were added to a mixer and mixed at a rotation speed of 15 rpm for 3 min. Then sieving was performed by using a 30-mesh screen, and the sieved material was mixed at a rotation speed of 15 rpm for 10 min. Magnesium stearate was sieved through a 30-mesh sieve and added to the mixer, and then mixing was performed at a rotation speed of 15 rpm for 3 min. The obtained mixed powders were filled into empty gelatin capsule shells to obtain oral capsules.

### EXAMPLE 8

### Detection of Properties of Mixed Powders

The micromeritic properties of the mixed powders obtained in Examples 4-7 and the solid dispersion powder of 5-fluoro-1-(4-fluoro-3-(4-(pyrimidin-2-yl)piperazine-1-carbonyl)benzyl)quinazoline-2,4(1H,3H)-dione were detected, and the results of angle of repose, water content, bulk density/tap density, Carr index, and the like are shown in Table 2.

TABLE 2
Detection results of micromeritic properties of mixed
powders in Examples 4-7 and solid dispersion powder

| | Bulk | Tap | Carr | | Water |
|---|---|---|---|---|---|
| | density | density | index | Angle of | content |
| Formulation | (g/mL) | (g/mL) | (%) | repose (°) | (%) |
| Example 4 | 0.41 | 0.62 | 33.9 | 45.1 | 2.73 |
| Example 5 | 0.39 | 0.61 | 36.1 | 48.1 | / |
| Example 6 | 0.40 | 0.60 | 33.3 | 45.9 | 2.02 |
| Example 7 | 0.41 | 0.61 | 32.8 | 43.9 | 2.05 |
| Solid dispersion | 0.22 | 0.43 | 48.8 | / | / |
| powder | | | | | |

As can be seen from the results in Table 2, the mixed powders in Examples 4-7 have significantly increased bulk density, significantly decreased Carl index, and significantly improved material fluidity compared with the solid dispersion powder.

In the formulations of Examples 4-7, the particle size (D90) of the filler (microcrystalline cellulose) is controlled to be 170-283 μm, and the particle size distribution of particles with a size of >75 μm (200 meshes) of the mannitol is controlled to be not less than 70%, and thus the fluidity of the material is effectively improved, and the capsule filling requirement is met.

### EXAMPLE 9

### Detection of Dissolution

The capsules prepared in Examples 4-7 and the direct-filling capsule of the solid dispersion powder of 5-fluoro-1-(4-fluoro-3-(4-(pyrimidin-2-yl)piperazine-1-carbonyl)benzyl)quinazoline-2,4(1H,3H)-dione (the formulation and preparation method of the solid dispersion powder were as described in Example 1) were subjected to detection of dissolution rate; the detection method was as follows: in the in vitro dissolution experiment, an automatic sampling dissolution tester was used for detection, the paddle method was selected, the water bath temperature of the automatic sampling dissolution tester was set to be 37±0.5° C., and a buffered solution with a pH of 6.8 and containing 2.0% SDS was selected as a dissolution medium, and the volume thereof was 900 mL. Samples were taken at 10 min, 15 min, 20 min, 30 min, 45 min and 60 min, and then taken after rotation at a limit speed of 250 rpm for 30 min, and all samples were passed through a nylon needle filter and analyzed by the determination method of sample dissolution rate. The results are shown in Tables 3-1 and 3-2.

TABLE 3-1

| | Example 4 | | Example 5 | | Example 6 | |
|---|---|---|---|---|---|---|
| | Average | | Average | | Average | |
| Time (min) | value (%) | RSD (%) | value (%) | RSD (%) | value (%) | RSD (%) |
| 10 | 46 | 45.8 | 64 | 23.2 | 48 | 59.0 |
| 15 | 61 | 22.9 | 72 | 14.7 | 73 | 29.0 |
| 20 | 77 | 19.4 | 79 | 7.0 | 86 | 15.4 |
| 30 | 87 | 8.3 | 88 | 5.6 | 92 | 6.6 |
| 45 | 92 | 4.9 | 90 | 6.0 | 97 | 4.1 |
| 60 | 94 | 3.5 | 92 | 5.1 | 100 | 2.7 |
| 90 | 99 | 3.1 | 95 | 5.5 | 103 | 2.2 |

TABLE 3-2

| | |
|---|---|
| | Solid dispersion powder |
| Example 7 | direct-filling capsule |

| Time | Average value | RSD | Average value | RSD |
|---|---|---|---|---|
| (min) | (%) | (%) | (%) | (%) |
| 10 | 65 | 18.3 | 70 | 13.3 |
| 15 | 82 | 11.4 | 80 | 9.9 |
| 20 | 88 | 4.7 | 85 | 13.0 |
| 30 | 94 | 2.6 | 83 | 9.2 |
| 45 | 98 | 1.9 | 86 | 9.7 |
| 60 | 101 | 1.7 | 89 | 9.9 |
| 90 | 103 | 1.2 | 98 | 2.0 |

The results in Tables 3-1 and 3-2 show that the capsules prepared in Examples 4-7 do not demonstrate significantly reduced dissolution rate compared to the solid dispersion powder direct-filling capsule, and all of them meet the quality control requirements of the product.

### EXAMPLE 10

### Preparation of Oral Capsule Comprising Solid Dispersion Powder of 5-fluoro-1-(4-fluoro-3-(4-(pyrimidin-2-yl)piperazine-1-carbonyl)benzyl)quinazoline-2,4(1H,3H)-dione

The formulation of capsule comprising 10 mg of 5-fluoro-1-(4-fluoro-3-(4-(pyrimidin-2-yl)piperazine-1-carbonyl)benzyl)quinazoline-2,4(1H,3H)-dione per capsule is shown below:

| Component | Weight (mg) |
|---|---|
| Solid dispersion powder of 5-fluoro-1-(4- | 40.00 |
| fluoro-3-(4-(pyrimidin-2-yl)piperazine-1- | |
| carbonyl)benzyl)quinazoline-2,4(1H,3H)-dione | |
| Microcrystalline cellulose PH200* | 61.04 |
| Mannitol 200SD** | 122.06 |
| Crospovidone XL*** | 1.15 |
| Colloidal silicon dioxide | 4.60 |
| Magnesium stearate | 1.15 |
| Empty gelatin capsules 0# | 1 capsule |
| Total amount | 230.00 |

*The particle size (D90) of the microcrystalline cellulose was controlled to be 275-480 μm.
**The particle size distribution of particles with a size of >75 μm (200 meshes) of the mannitol should be controlled to be not less than 90%.
***The particle size (D90) of the crospovidone is controlled to be 270-385 μm.

Preparation Method:

The solid dispersion powder of 5-fluoro-1-(4-fluoro-3-(4-(pyrimidin-2-yl)piperazine-1-carbonyl)benzyl)quinazoline-2,4(1H,3H)-dione prepared in Example 1, microcrystalline cellulose, mannitol, crospovidone, and colloidal silicon dioxide were added to a hopper mixer and mixed at a rotation speed of 15 rpm for 3 min. Then sieving was performed by using a 30-mesh screen, and the sieved material was mixed at a rotation speed of 15 rpm for 10 min. Magnesium stearate was sieved through a 30-mesh sieve and added to the mixer, and then mixing was performed at a rotation speed of 15 rpm for 3 min. The obtained mixed powders were filled into empty gelatin capsule shells to obtain oral capsules.

### EXAMPLE 11

### Preparation of Oral Capsule Comprising Solid Dispersion Powder of 5-fluoro-1-(4-fluoro-3-(4-(pyrimidin-2-yl)piperazine-1-carbonyl)benzyl)quinazoline-2,4(1H,3H)-dione

The formulation of capsule comprising 10 mg of 5-fluoro-1-(4-fluoro-3-(4-(pyrimidin-2-yl)piperazine-1-carbonyl)benzyl)quinazoline-2,4(1H,3H)-dione per capsule is shown below:

| Component | Weight (mg) |
|---|---|
| Solid dispersion powder of 5-fluoro-1-(4- | 40.00 |
| fluoro-3-(4-(pyrimidin-2-yl)piperazine-1- | |
| carbonyl)benzyl)quinazoline-2,4(1H,3H)-dione | |
| Microcrystalline cellulose PH200* | 60.26 |
| Mannitol 200SD** | 120.54 |
| Crospovidone XL*** | 1.15 |
| Colloidal silicon dioxide | 6.90 |
| Magnesium stearate | 1.15 |
| Empty gelatin capsules 0# | 1 capsule |
| Total amount | 230.00 |

*The particle size (D90) of the microcrystalline cellulose was controlled to be 275-480 μm.
**The particle size distribution of particles with a size of >75 μm (200 meshes) of the mannitol should be controlled to be not less than 90%.
***The particle size (D90) of the crospovidone is controlled to be 270-385 μm.

Preparation Method:

The solid dispersion powder of 5-fluoro-1-(4-fluoro-3-(4-(pyrimidin-2-yl)piperazine-1-carbonyl)benzyl)quinazoline-2,4(1H,3H)-dione prepared in Example 1, microcrystalline cellulose, mannitol, crospovidone, and colloidal silicon dioxide were added to a mixer and mixed at a rotation speed of 15 rpm for 3 min. Then sieving was performed by using a 30-mesh screen, and the sieved material was mixed at a rotation speed of 15 rpm for 10 min. Magnesium stearate was sieved through a 30-mesh sieve and added to the mixer, and then mixing was performed at a rotation speed of 15 rpm for 3 min. The obtained mixed powders were filled into empty gelatin capsule shells to obtain oral capsules.

### EXAMPLE 12

### Preparation of Oral Capsule Comprising Solid Dispersion Powder of 5-fluoro-1-(4-fluoro-3-(4-(pyrimidin-2-yl)piperazine-1-carbonyl)benzyl)quinazoline-2,4(1H,3H)-dione

The formulation of capsule comprising 10 mg of 5-fluoro-1-(4-fluoro-3-(4-(pyrimidin-2-yl)piperazine-1-carbonyl)benzyl)quinazoline-2,4(1H,3H)-dione per capsule is shown below:

| Component | Weight (mg) |
|---|---|
| Solid dispersion powder of 5-fluoro-1-(4- | 40.00 |
| fluoro-3-(4-(pyrimidin-2-yl)piperazine-1- | |
| carbonyl)benzyl)quinazoline-2,4(1H,3H)-dione | |
| Microcrystalline cellulose PH200* | 59.89 |
| Mannitol 200SD** | 119.76 |
| Crospovidone XL*** | 2.30 |
| Colloidal silicon dioxide | 6.90 |
| Magnesium stearate | 1.15 |
| Empty gelatin capsules 0# | 1 capsule |
| Total amount | 230.00 |

*The particle size (D90) of the microcrystalline cellulose was controlled to be 275-480 μm.
**The particle size distribution of particles with a size of >75 μm (200 meshes) of the mannitol should be controlled to be not less than 90%.
***The particle size (D90) of the crospovidone is controlled to be 270-385 μm.

Preparation Method:

The solid dispersion powder of 5-fluoro-1-(4-fluoro-3-(4-(pyrimidin-2-yl)piperazine-1-carbonyl)benzyl)quinazoline-2,4(1H,3H)-dione prepared in Example 1, microcrystalline cellulose, mannitol, crospovidone, and colloidal silicon dioxide were added to a mixer and mixed at a rotation speed of 15 rpm for 3 min. Then sieving was performed by using a 30-mesh screen, and the sieved material was mixed at a rotation speed of 15 rpm for 10 min. Magnesium stearate was sieved through a 30-mesh sieve and added to the mixer, and then mixing was performed at a rotation speed of 15 rpm for 3 min. The obtained mixed powders were filled into empty gelatin capsule shells to obtain oral capsules.

### EXAMPLE 13

### Preparation of Oral Capsule Comprising Solid Dispersion Powder of 5-fluoro-1-(4-fluoro-3-(4-(pyrimidin-2-yl)piperazine-1-carbonyl)benzyl)quinazoline-2,4(1H,3H)-dione

The formulation of capsule comprising 10 mg of 5-fluoro-1-(4-fluoro-3-(4-(pyrimidin-2-yl)piperazine-1-carbonyl)benzyl)quinazoline-2,4(1H,3H)-dione per capsule is shown below:

| Component | Weight (mg) |
|---|---|
| Solid dispersion powder of 5-fluoro-1-(4- | 40.00 |
| fluoro-3-(4-(pyrimidin-2-yl)piperazine-1- | |
| carbonyl)benzyl)quinazoline-2,4(1H,3H)-dione | |
| Microcrystalline cellulose PH200* | 91.55 |
| Mannitol 200SD** | 91.55 |
| Crospovidone XL*** | 1.15 |
| Colloidal silicon dioxide | 4.6 |
| Magnesium stearate | 1.15 |
| Empty gelatin capsules 0# | 1 capsule |
| Total amount | 230.00 |

*The particle size (D90) of the microcrystalline cellulose was controlled to be 275-480 μm.
**The particle size distribution of particles with a size of >75 μm (200 meshes) of the mannitol should be controlled to be not less than 90%.
***The particle size (D90) of the crospovidone is controlled to be 270-385 μm.

Preparation Method:

The solid dispersion powder of 5-fluoro-1-(4-fluoro-3-(4-(pyrimidin-2-yl)piperazine-1-carbonyl)benzyl)quinazoline-2,4(1H,3H)-dione, microcrystalline cellulose, mannitol, crospovidone, and colloidal silicon dioxide were added to a mixer and mixed at a rotation speed of 6 rpm for 3 min. Then sieving was performed by using a 30-mesh screen, and the sieved material was mixed at a rotation speed of 6 rpm for 10 min. Magnesium stearate was sieved through a 30-mesh sieve and added to the mixer, and then mixing was performed at a rotation speed of 6 rpm for 3 min. The obtained mixed powders were filled into empty gelatin capsule shells to obtain oral capsules.

### EXAMPLE 14

### Preparation of Oral Capsule Comprising Solid Dispersion Powder of 5-fluoro-1-(4-fluoro-3-(4-(pyrimidin-2-yl)piperazine-1-carbonyl)benzyl)quinazoline-2,4(1H,3H)-dione

The formulation of capsule comprising 10 mg of 5-fluoro-1-(4-fluoro-3-(4-(pyrimidin-2-yl)piperazine-1-carbonyl)benzyl)quinazoline-2,4(1H,3H)-dione per capsule is shown below:

| Component | Weight (mg) |
|---|---|
| Solid dispersion powder of 5-fluoro-1-(4- | 40.00 |
| fluoro-3-(4-(pyrimidin-2-yl)piperazine-1- | |
| carbonyl)benzyl)quinazoline-2,4(1H,3H)-dione | |
| Microcrystalline cellulose PH200* | 122.06 |
| Mannitol 200SD** | 61.04 |
| Crospovidone XL*** | 1.15 |
| Colloidal silicon dioxide | 4.6 |
| Magnesium stearate | 1.15 |
| Empty gelatin capsules 0# | 1 capsule |
| Total amount | 230.00 |

*The particle size (D90) of the microcrystalline cellulose was controlled to be 275-480 μm.
**The particle size distribution of particles with a size of >75 μm (200 meshes) of the mannitol should be controlled to be not less than 90%.
***The particle size (D90) of the crospovidone is controlled to be 270-385 μm.

Preparation Method:

The solid dispersion powder of 5-fluoro-1-(4-fluoro-3-(4-(pyrimidin-2-yl)piperazine-1-carbonyl)benzyl)quinazoline-2,4(1H,3H)-dione, microcrystalline cellulose, mannitol, crospovidone, and colloidal silicon dioxide were added to a mixer and mixed at a rotation speed of 6 rpm for 3 min. Then sieving was performed by using a 30-mesh screen, and the sieved material was mixed at a rotation speed of 6 rpm for 10 min. Magnesium stearate was sieved through a 30-mesh sieve and added to the mixer, and then mixing was performed at a rotation speed of 6 rpm for 3 min. The obtained mixed powders were filled into empty gelatin capsule shells to obtain oral capsules.

### EXAMPLE 15

### Detection of Properties of Mixed Powders

The micromeritic properties of the mixed powders obtained in Examples 10-14 and the solid dispersion powder of 5-fluoro-1-(4-fluoro-3-(4-(pyrimidin-2-yl)piperazine-1-carbonyl)benzyl)quinazoline-2,4(1H,3H)-dione were detected, and the results of angle of repose, water content, bulk density/tap density, Carr index, and the like are shown in Table 4.

TABLE 4
Detection results of micromeritic properties of mixed
powders in Examples 10-14 and solid dispersion powders

| | Bulk | Tap | Carr | Angle of | Water |
|---|---|---|---|---|---|
| | density | density | index | repose | content |
| Formula | (g/mL) | (g/mL) | (%) | (°) | (%) |
| Example 10 | 0.38 | 0.61 | 37.7 | 44.0 | 1.34 |
| Example 11 | 0.36 | 0.56 | 35.7 | 44.7 | 1.74 |
| Example 12 | 0.37 | 0.55 | 32.7 | 41.3 | 1.51 |
| Example 13 | 0.37 | 0.57 | 35.1 | 44.8 | 2.90 |
| Example 14 | 0.35 | 0.55 | 36.4 | 41.7 | 3.50 |
| Solid dispersion | 0.22 | 0.43 | 48.8 | / | / |
| powder | | | | | |

As can be seen from the results in Table 4, the mixed powders in Examples 10-14 demonstrate significantly increased bulk density, significantly decreased Carl index, and significantly improved material fluidity compared with the solid dispersion powder.

### EXAMPLE 16

### Capsule Filling Study

The mixed powders prepared in Examples 10-14 were subjected to capsule filling by using a lab-scale automatic capsule filling machine. After the machine was commissioned to reach a target filling weight, formal capsule filling was performed and the weight of the capsules was detected. The detection results are shown in Tables 5-8.

TABLE 5
Capsule weighing results during filling of the mixed powders prepared in Example 10

| Formula | | Example 10 | | | | | | | | |
|---|---|---|---|---|---|---|---|---|---|---|
| Single | 1 | 321 | 320 | 324 | 343 | 336 | 342 | 327 | 316 | 323 |
| capsule | 2 | 324 | 319 | 328 | 341 | 339 | 329 | 337 | 325 | 323 |
| Weight | 3 | 326 | 339 | 328 | 326 | 331 | 333 | 343 | 323 | 310 |
| (mg)* | 4 | 336 | 319 | 313 | 330 | 340 | 327 | 338 | 343 | 319 |
| | 5 | 332 | 327 | 330 | 338 | 328 | 334 | 328 | 332 | 325 |
| | 6 | 329 | 322 | 317 | 322 | 327 | 342 | 341 | 328 | 320 |
| | 7 | 324 | 331 | 321 | 321 | 328 | 328 | 325 | 330 | 328 |
| | 8 | 331 | 315 | 318 | 315 | 323 | 327 | 326 | 324 | 325 |
| | 9 | 324 | 335 | 336 | 310 | 341 | 343 | 322 | 320 | 317 |
| | 10 | 322 | 338 | 332 | 335 | 326 | 322 | 327 | 321 | 327 |

| | | | | | | | | | |
|---|---|---|---|---|---|---|---|---|---|
| Average weight | 326.9 | 326.5 | 324.7 | 328.1 | 331.9 | 332.7 | 331.4 | 326.2 | 321.7 |
| of 10 capsules (mg)** | | | | | | | | | |
| Relative standard | 1.49 | 2.68 | 2.26 | 3.41 | 1.97 | 2.23 | 2.27 | 2.33 | 1.68 |
| deviation of 10 | | | | | | | | | |
| capsules (RSD < 5%) | | | | | | | | | |

*Upper limit: 345.27; Target: 328.02; Lower limit: 310.77;
**Upper limit: 339.52; Lower limit: 316.52.

TABLE 6
Capsule weighing results during filling of
the mixed powders prepared in Example 11

| Formula | | Example 11 | | | | | | |
|---|---|---|---|---|---|---|---|---|
| Single | 1 | 331.8 | 336.0 | 333.7 | 325.6 | 336.7 | 317.8 | 313.9 |
| capsule | 2 | 331.8 | 333.9 | 328.2 | 326.2 | 328.9 | 321.6 | 323.1 |
| Weight | 3 | 334.7 | 330.6 | 323.2 | 328.4 | 335.1 | 318.7 | 322.2 |
| (mg)* | 4 | 325.7 | 324.8 | 331.2 | 330.5 | 325.9 | 317.7 | 321.1 |
| | 5 | 322.4 | 333.8 | 327.4 | 332.1 | 334.9 | 320.0 | 313.1 |
| | 6 | 327.0 | 329.9 | 332.2 | 323.9 | 322.4 | 314.9 | 313.5 |
| | 7 | 331.0 | 334.6 | 335.7 | 324.8 | 329.9 | 319.7 | 319.9 |
| | 8 | 327.6 | 335.8 | 327.9 | 328.1 | 328.4 | 318.0 | 313.5 |
| | 9 | 334.4 | 332.0 | 323.4 | 325.5 | 330.1 | 315.6 | 323.8 |
| | 10 | 322.2 | 323.9 | 325.2 | 324.8 | 329.3 | 319.5 | 330.0 |

| | | | | | | | |
|---|---|---|---|---|---|---|---|
| Average weight of 10 | 328.86 | 331.53 | 328.82 | 326.99 | 330.16 | 318.35 | 319.41 |
| capsules (mg)** | | | | | | | |
| Relative standard | 1.39 | 1.29 | 1.30 | 0.83 | 1.33 | 0.64 | 1.80 |
| deviation of 10 | | | | | | | |
| capsules (RSD < 5%) | | | | | | | |

*Upper limit: 345.27; Target: 328.02; Lower limit: 310.77;
**Upper limit: 339.52; Lower limit: 316.52.

TABLE 7
Capsule weighing results during filling of
the mixed powders prepared in Example 12

| Formula | | Example 12 | | | | | | |
|---|---|---|---|---|---|---|---|---|
| Single | 1 | 334 | 324 | 335 | 332 | 332 | 329 | 317 |
| capsule | 2 | 321 | 331 | 335 | 331 | 331 | 321 | 317 |
| Weight | 3 | 328 | 327 | 325 | 323 | 332 | 330 | 307 |
| (mg)* | 4 | 328 | 329 | 327 | 327 | 329 | 329 | 322 |
| | 5 | 323 | 324 | 327 | 332 | 338 | 333 | 323 |
| | 6 | 326 | 335 | 328 | 334 | 334 | 329 | 321 |
| | 7 | 323 | 332 | 334 | 329 | 322 | 333 | 320 |
| | 8 | 324 | 329 | 335 | 323 | 327 | 330 | 318 |
| | 9 | 330 | 330 | 329 | 325 | 335 | 329 | 320 |
| | 10 | 319 | 329 | 336 | 319 | 323 | 330 | 317 |

| | | | | | | | |
|---|---|---|---|---|---|---|---|
| Average weight of 10 | 325.6 | 329.0 | 331.1 | 327.5 | 330.3 | 329.3 | 318.2 |
| capsules (mg)** | | | | | | | |
| Relative standard | 1.38 | 1.03 | 1.28 | 1.50 | 1.55 | 1.0 | 1.41 |
| deviation of 10 capsules | | | | | | | |
| (RSD < 5%) | | | | | | | |

*Upper limit: 345.27; Target: 328.02; Lower limit: 310.77;
**Upper limit: 339.52; Lower limit: 316.52.

TABLE 8
Capsule weighing results during filling of the
mixed powders prepared in Examples 13-14

| Formula | | Example 13 | | | Example 14 | | |
|---|---|---|---|---|---|---|---|
| Weight of | 1 | 324.8 | 329.2 | 325.9 | 326.7 | 328.3 | 322.6 |
| single capsule | 2 | 326.7 | 329.5 | 326.2 | 327.4 | 326.3 | 320.3 |
| (mg)* | 3 | 326.7 | 328.2 | 327.3 | 324.8 | 329.4 | 328.4 |
| | 4 | 325.9 | 332.3 | 326.6 | 327.8 | 329.8 | 326.0 |
| | 5 | 325.6 | 327.8 | 326.3 | 326.1 | 324.9 | 323.5 |
| | 6 | 327.8 | 331.3 | 325.3 | 334.6 | 324.5 | 327.3 |
| | 7 | 323.4 | 328.5 | 330.3 | 328.6 | 326.5 | 322.7 |
| | 8 | 323.0 | 328.5 | 325.2 | 325.5 | 325.3 | 322.7 |
| | 9 | 325.6 | 329.1 | 325.0 | 322.4 | 330.7 | 326.1 |
| | 10 | 322.8 | 329.8 | 325.9 | 322.0 | 330.2 | 327.6 |

| | | | | | | |
|---|---|---|---|---|---|---|
| Average weight of 10 | 325.23 | 329.42 | 326.4 | 326.59 | 327.59 | 324.72 |
| capsules (mg)** | | | | | | |
| Relative standard | 0.52 | 0.43 | 0.47 | 1.09 | 0.72 | 0.83 |
| deviation of 10 capsules | | | | | | |
| (RSD < 5%) | | | | | | |

*Upper limit: 343.72; Target: 326.47; Lower limit: 309.22;
**Upper limit: 337.97; Lower limit: 314.97.

Capsule weighing in-process control for capsule filling process was performed every 10 minutes for each of Examples 10-14. The results in Tables 5-8 show that all the capsule weights are within limits and have a small relative standard deviation in the process of capsule filling for 70 min of Examples 10-12 (for 30 min of Examples 13-14). The results show that the types and the proportions of the excipients in Examples 10-14 effectively improve the fluidity of the materials, and thus the requirements for equipment encapsulation can be met.

### EXAMPLE 17

### Detection of Dissolution

The capsules prepared in Examples 10-14 and the direct-filling capsule of the solid dispersion powder of 5-fluoro-1-(4-fluoro-3-(4-(pyrimidin-2-yl)piperazine-1-carbonyl)benzyl)quinazoline-2,4(1H,3H)-dione (the formulation and preparation method of the solid dispersion powder were as described in Example 1) were subjected to detection of dissolution rate; the detection method was as follows: in the in vitro dissolution experiment, an automatic sampling dissolution tester was used for detection, the basket method in the section “Dissolution Rate” of Chinese Pharmacopoeia 0931 was selected, the water bath temperature of the automatic sampling dissolution tester was set to be 37±0.5° C., and a buffered solution with a pH of 6.8 and containing 2.0% SDS was selected as a dissolution medium, and the volume thereof was 900 mL. Samples were taken at 10 min, 15 min, 20 min, 30 min, 45 min and 60 min, and then taken after rotation at a limit speed of 250 rpm for 30 min, and all samples were passed through a nylon needle filter and analyzed by the determination method of sample dissolution rate. The results are shown in Table 9.

TABLE 9
Dissolution of capsules prepared in Examples 10-14 and solid dispersion powder direct-filling capsule

| | | | | | | | | | | | Solid | |
|---|---|---|---|---|---|---|---|---|---|---|---|---|
| | | | | | | | | | | | dispersion | |
| | | | | | | | | | | | powder | |
| | | | | | | | | | | | direct-filling | |
| | Example 10 | | Example 11 | | Example 12 | | Example 13 | | Example 14 | | capsule | |
| | Average | | Average | | Average | | Average | | Average | | Average | |
| Time | value | RSD | value | RSD | value | RSD | value | RSD | value | RSD | value | RSD |
| (min) | (%) | (%) | (%) | (%) | (%) | (%) | (%) | (%) | (%) | (%) | (%) | (%) |
| 10 | 63 | 30.9 | 51 | 22.3 | 61 | 41.0 | 43 | 20.9 | 55 | 32.2 | 70 | 13.3 |
| 15 | 69 | 26.1 | 59 | 20.6 | 68 | 32.0 | 46 | 23.8 | 57 | 31.0 | 80 | 9.9 |
| 20 | 76 | 17.2 | 68 | 12.1 | 76 | 21.0 | 55 | 18.2 | 60 | 28.6 | 85 | 13.0 |
| 30 | 86 | 8.6 | 79 | 5.3 | 84 | 11.1 | 70 | 6.8 | 66 | 26.4 | 83 | 9.2 |
| 45 | 93 | 3.1 | 89 | 2.1 | 91 | 5.2 | 82 | 3.4 | 76 | 21.0 | 86 | 9.7 |
| 60 | 96 | 1.5 | 93 | 1.9 | 94 | 3.3 | 88 | 2.4 | 82 | 17.4 | 89 | 9.9 |
| 90 | 100 | 1.6 | 98 | 2.4 | 98 | 1.6 | 97 | 1.8 | 98 | 1.3 | 98 | 2.0 |

The results in Table 9 show that the capsules prepared in Examples 10-14 do not demonstrate a significantly reduced dissolution speed compared to the solid dispersion powder direct-filling capsule.

### EXAMPLE 18

### Detection of Content Uniformity

The capsules prepared in Examples 10-12 were detected for content uniformity and the results are shown in Table 10.

TABLE 10
Detection results of content uniformity of capsules prepared in Examples 10-12

| Example 10 | | | Example 11 | | | Example 12 | | |
|---|---|---|---|---|---|---|---|---|
| | Average | | | Average | | | Average | |
| | content | RSD | Content | content | RSD | Content | content | RSD |
| Content (%) | (%) | (%) | (%) | (%) | (%) | (%) | (%) | (%) |
| 99.812 | 100.5 | 2.3 | 95.922 | 98.7 | 2.6 | 100.474 | 96.4 | 2.2 |
| 99.626 | | | 102.045 | | | 95.263 | | |
| 102.553 | | | 97.451 | | | 97.109 | | |
| 100.571 | | | 95.977 | | | 94.202 | | |
| 103.889 | | | 97.954 | | | 94.997 | | |
| 103.381 | | | 102.748 | | | 94.077 | | |
| 98.062 | | | 96.769 | | | 95.908 | | |
| 96.470 | | | 98.794 | | | 96.679 | | |
| 100.614 | | | 101.664 | | | 99.475 | | |
| 99.649 | | | 98.160 | | | 95.845 | | |

| | | | | | |
|---|---|---|---|---|---|
| A + 2.2S (%) | 5.6 | A + 2.2S (%) | 6.8 | A + 2.2S (%) | 8.3 |
| Limit of not more | Comply with | Limit of not more | Comply with | Limit of not more | Comply with |
| than 15 | specification | than 15 | specification | than 15 | specification |

The results in Table 10 show that the content uniformity of the capsules in Examples 10-12 complies with specification.

In the formulations of Examples 10-14, the particle size (D90) of the filler (microcrystalline cellulose) is 275-480 μm, and the particle size distribution of particles with a size of >75 μm (200 meshes) of the mannitol is controlled to be not less than 90%, resulting in that the particle size of the filler is almost doubled compared with that of the filler in Examples 2 and 4-7. The material fluidity and the capsule filling adaptability on automatic encapsulation equipment are further improved. Meanwhile it has been surprisingly found that the increase in the particle size of excipients does not lead to the occurrence of delamination, uneven mixing, or the like during the mixing and capsule filling process. Furthermore, the scaled-up trial production results indicate an excellent uniformity of the mixed materials and content uniformity of the finished capsule product, suggesting that the formulations could well overcome the defects of the physicochemical properties of the solid dispersion powder and have the required dissolution characteristic, meanwhile could meet the requirements of the direct-mixing and capsule filling process.

### EXAMPLE 19

### PK Study in Dogs

The capsules prepared in Example 10 and the direct-filling capsule of the solid dispersion powder of 5-fluoro-1-(4-fluoro-3-(4-(pyrimidin-2-yl)piperazine-1-carbonyl) benzyl)quinazoline-2,4(1H,3H)-dione (the formulation and preparation method of the solid dispersion powder were as described in Example 1) were subjected to in vivo PK experimental study in dogs. All experimental dogs were fasted and fed 4 hours after administration. The administration dose was 0.8 mpk. The results are shown in Table 11.

TABLE 11
PK test results for capsules prepared in Example 10
and solid dispersion powder direct-filling capsules

| | Solid dispersion powder | | | |
|---|---|---|---|---|
| Formula | direct-filling capsule | | Example 10 | |
| PK parameters | Average value | SD | Average value | SD |
| Cₘₐₓ (ng/ml) | 172 | 90.0 | 126 | 72.3 |
| Tₘₐₓ (h) | 0.861 | 0.636 | 1.22 | 0.656 |
| T_{1/2} (h) | 3.81 | 2.64 | 2.26 | 0.350 |
| Tₗₐₛₜ (h) | ND* | — | 12.0 | — |
| AUC₀₋ₗₐₛₜ (ng · h/ml) | 519 | 238 | 424 | 208 |
| AUC_{0-inf} (ng · h/ml) | 531 | 242 | 435 | 211 |
| MRT₀₋ₗₐₛₜ (h) | 3.25 | 1.06 | 3.25 | 0.449 |
| MRT_{0-inf} (h) | 3.78 | 1.57 | 3.57 | 0.493 |
| AUC_{Extra} (%) | 2.33 | 1.28 | 2.68 | 0.900 |
| AUMC_{Extra} (%) | 13.4 | 7.97 | 11.4 | 3.45 |

*ND represents that it cannot be determined because only less than 2 data values are present.

According to analysis of the data in Table 11, the Cₘₐₓ and AUC₀₋ₗₐₛₜ of the capsules prepared in Example 10 were not significantly different from those of the solid dispersion powder direct-filling capsules.

### EXAMPLE 20

Multiple batches of scaled-up production were performed using the formulation in Example 10 until the commercial batch (1,000,000 capsules) was reached, and some representative batches produced are summarized herein in Table 12.

TABLE 12
Scaled-up representative batches' information in Example 10

| Batch No. | Batch | |
|---|---|---|
| A | 275,000 | capsules |
| B | 400,000 | capsules |
| C | 1,000,000 | capsules |

Preparation method: The solid dispersion powder of 5-fluoro-1-(4-fluoro-3-(4-(pyrimidin-2-yl)piperazine-1-carbonyl)benzyl)quinazoline-2,4(1H,3H)-dione prepared in Example 1, microcrystalline cellulose, mannitol, crospovidone, and colloidal silicon dioxide were added to a hopper blender and mixed at a rotation speed of 6 rpm for 3 min. Then sieving was performed by using a 30-mesh screen, and the sieved material was mixed at a rotation speed of 6 rpm for 10 min. Magnesium stearate was sieved through a 30-mesh sieve and added to the hopper mixer, and then mixing was performed at a rotation speed of 6 rpm for 3 min. The obtained mixed powders were filled into empty gelatin capsule shells to obtain oral capsules.

The mixed powder of each batch in Example 20 has good blending uniformity and fluidity, and thus smooth filling of capsules can be achieved. The results of blending uniformity, bulk density/tap density and Carr index are shown in Table 13.

TABLE 13
Blending uniformity, bulk density, tap density
and Carr index of mixed powders of Example 20

| | Blending uniformity | | Bulk | Tap | |
|---|---|---|---|---|---|
| Batch | Average | | density/ | density/ | Carr |
| No. | value/% | RSD/% | g/mL | g/mL | index/% |
| A | 100.3 | 1.1 | 0.396 | 0.622 | 36.33 |
| B | 98.2 | 1.6 | 0.378 | 0.606 | 37.62 |
| C | 99.0 | 2.0 | 0.41 | 0.63 | 34.92 |

Oral capsules batches in Example 20 were investigated for dissolution, and the results show that the dissolution rates at 60 min were all greater than 75%, and thus the capsules can meet the requirements for quality control.

Oral capsules comprising the solid dispersion powder of 5-fluoro-1-(4-fluoro-3-(4-(pyrimidin-2-yl)piperazine-1-carbonyl)benzyl)quinazoline-2,4(1H,3H)-dione were packaged in high-density polyethylene bottles, sealed and investigated for stability under the conditions of 25° C./60% RH and 40° C./75% RH, the detection items included content, related substances, crystalline forms and the like. The results show that the capsules are stable for 24 months.

### EXAMPLE 21

### Preparation of Oral Capsule Comprising Solid Dispersion Powder of 5-fluoro-1-(4-fluoro-3-(4-(pyrimidin-2-yl)piperazine-1-carbonyl)benzyl)quinazoline-2,4(1H,3H)-dione

The formulation of capsules comprising 20 mg of 5-fluoro-1-(4-fluoro-3-(4-(pyrimidin-2-yl)piperazine-1-carbonyl)benzyl)quinazoline-2,4(1H,3H)-dione per capsule is shown below:

| Component | Weight (mg) |
|---|---|
| Solid dispersion powder of 5-fluoro-1-(4- | 80.00 |
| fluoro-3-(4-(pyrimidin-2-yl)piperazine-1- | |
| carbonyl)benzyl)quinazoline-2,4(1H,3H)-dione | |
| Microcrystalline cellulose PH200* | 109.12 |
| Mannitol 200SD** | 218.27 |
| Crospovidone XL*** | 2.10 |
| Colloidal silicon dioxide | 8.40 |
| Magnesium stearate | 2.10 |
| Empty gelatin capsules 0#el | 1 capsule |
| Total amount | 420.00 |

*The particle size (D90) of the microcrystalline cellulose was controlled to be 275-480 μm.
**The particle size distribution of particles with a size of >75 μm (200 meshes) of the mannitol should be controlled to be not less than 90%.
***The particle size (D90) of the crospovidone is controlled to be 270-385 μm.

Preparation Method:

The solid dispersion powder of 5-fluoro-1-(4-fluoro-3-(4-(pyrimidin-2-yl)piperazine-1-carbonyl)benzyl)quinazoline-2,4(1H,3H)-dione prepared in Example 1, microcrystalline cellulose, mannitol, crospovidone, and colloidal silicon dioxide were added to a mixer and mixed at a rotation speed of 15 rpm for 3 min. Then sieving was performed by using a 30-mesh screen, and the sieved material was mixed at a rotation speed of 15 rpm for 10 min. Magnesium stearate was sieved through a 30-mesh sieve and added to the mixer, and then mixing was performed at a rotation speed of 15 rpm for 3 min. The obtained mixed powders were filled into empty gelatin capsule shells to obtain oral capsules.

### EXAMPLE 22

### Preparation of Oral Capsule Comprising Solid Dispersion Powder of 5-fluoro-1-(4-fluoro-3-(4-(pyrimidin-2-yl)piperazine-1-carbonyl)benzyl)quinazoline-2,4(1H,3H)-dione

The formulation of capsules comprising 20 mg of 5-fluoro-1-(4-fluoro-3-(4-(pyrimidin-2-yl)piperazine-1-carbonyl)benzyl)quinazoline-2,4(1H,3H)-dione per capsule is shown below:

| Component | Weight (mg) |
|---|---|
| Solid dispersion powder of 5-fluoro-1-(4- | 80.00 |
| fluoro-3-(4-(pyrimidin-2-y1)piperazine-1- | |
| carbonyl)benzyl)quinazoline-2,4(1H,3H)-dione | |
| Microcrystalline cellulose PH200* | 42.00 |
| Mannitol 200SD** | 285.39 |
| Crospovidone XL*** | 2.10 |
| Colloidal silicon dioxide | 8.40 |
| Magnesium stearate | 2.10 |
| Empty gelatin capsules 0#el | 1 capsule |
| Total amount | 420.00 |

*The particle size (D90) of the microcrystalline cellulose was controlled to be 275-480 μm.
**The particle size distribution of particles with a size of >75 μm (200 meshes) of the mannitol should be controlled to be not less than 90%.
***The particle size (D90) of the crospovidone is controlled to be 270-385 μm.

Preparation Method:

The solid dispersion powder of 5-fluoro-1-(4-fluoro-3-(4-(pyrimidin-2-yl)piperazine-1-carbonyl)benzyl)quinazoline-2,4(1H,3H)-dione prepared in Example 1, microcrystalline cellulose, mannitol, crospovidone, and colloidal silicon dioxide were added to a mixer and mixed at a rotation speed of 15 rpm for 3 min. Then sieving was performed by using a 30-mesh screen, and the sieved material was mixed at a rotation speed of 15 rpm for 10 min. Magnesium stearate was sieved through a 30-mesh sieve and added to the mixer, and then mixing was performed at a rotation speed of 15 rpm for 3 min. The obtained mixed powders were filled into empty gelatin capsule shells to obtain oral capsules.

### EXAMPLE 23

### Preparation of Oral Capsule Comprising Solid Dispersion Powder of 5-fluoro-1-(4-fluoro-3-(4-(pyrimidin-2-yl)piperazine-1-carbonyl)benzyl)quinazoline-2,4(1H,3H)-dione

The formulation of capsules comprising 20 mg of 5-fluoro-1-(4-fluoro-3-(4-(pyrimidin-2-yl)piperazine-1-carbonyl)benzyl)quinazoline-2,4(1H,3H)-dione per capsule is shown below:

| Component | Weight (mg) |
|---|---|
| Solid dispersion powder of 5-fluoro-1-(4- | 80.00 |
| fluoro-3-(4-(pyrimidin-2-yl)piperazine-1- | |
| carbonyl)benzyl)quinazoline-2,4(1H,3H)-dione | |
| Microcrystalline cellulose PH200* | 63.00 |
| Mannitol 200SD** | 258.09 |
| Crospovidone XL*** | 8.40 |
| Colloidal silicon dioxide | 8.40 |
| Sodium fumarate | 2.10 |
| Empty gelatin capsules 0#el | 1 capsule |
| Total amount | 420.00 |

*The particle size (D90) of the microcrystalline cellulose was controlled to be 275-480 μm.
**The particle size distribution of particles with a size of >75 μm (200 meshes) of the mannitol should be controlled to be not less than 90%.
***The particle size (D90) of the crospovidone is controlled to be 270-385 μm.

Preparation Method:

The solid dispersion powder of 5-fluoro-1-(4-fluoro-3-(4-(pyrimidin-2-yl)piperazine-1-carbonyl)benzyl)quinazoline-2,4(1H,3H)-dione prepared in Example 1, microcrystalline cellulose, mannitol, crospovidone, and colloidal silicon dioxide were added to a mixer and mixed at a rotation speed of 15 rpm for 3 min. Then sieving was performed by using a 30-mesh screen, and the sieved material was mixed at a rotation speed of 15 rpm for 10 min. Sodium fumarate was sieved through a 30-mesh sieve and added to the mixer, and then mixing was performed at a rotation speed of 15 rpm for 3 min. The obtained mixed powders were filled into empty gelatin capsule shells to obtain oral capsules.

### EXAMPLE 24

### Preparation of Oral Capsule Comprising Solid Dispersion Powder of 5-fluoro-1-(4-fluoro-3-(4-(pyrimidin-2-yl)piperazine-1-carbonyl)benzyl)quinazoline-2,4(1H,3H)-dione

The formulation of capsules comprising 20 mg of 5-fluoro-1-(4-fluoro-3-(4-(pyrimidin-2-yl)piperazine-1-carbonyl)benzyl)quinazoline-2,4(1H,3H)-dione per capsule is shown below:

| Component | Weight (mg) |
|---|---|
| Solid dispersion powder of 5-fluoro-1-(4- | 80.00 |
| fluoro-3-(4-(pyrimidin-2-yl)piperazine-1- | |
| carbonyl)benzyl)quinazoline-2,4(1H,3H)-dione | |
| Microcrystalline cellulose PH200* | 63.84 |
| Mannitol 200SD** | 258.1 |
| Crospovidone XL*** | 8.40 |
| Colloidal silicon dioxide | 8.40 |
| Magnesium stearate | 1.26 |
| Empty gelatin capsules 0#el | 1 capsule |
| Total amount | 420.00 |

*The particle size (D90) of the microcrystalline cellulose was controlled to be 275-480 μm.
**The particle size distribution of particles with a size of >75 μm (200 meshes) of the mannitol should be controlled to be not less than 90%.
***The particle size (D90) of the crospovidone is controlled to be 270-385 μm.

Preparation Method:

The solid dispersion powder of 5-fluoro-1-(4-fluoro-3-(4-(pyrimidin-2-yl)piperazine-1-carbonyl)benzyl)quinazoline-2,4(1H,3H)-dione prepared in Example 1, microcrystalline cellulose, mannitol, crospovidone, and colloidal silicon dioxide were added to a mixer and mixed at a rotation speed of 15 rpm for 3 min. Then sieving was performed by using a 30-mesh screen, and the sieved material was mixed at a rotation speed of 15 rpm for 10 min. Magnesium stearate was sieved through a 30-mesh sieve and added to the mixer, and then mixing was performed at a rotation speed of 15 rpm for 3 min. The obtained mixed powders were filled into empty gelatin capsule shells to obtain oral capsules.

It is detected using the method described above that the mixed powders prepared in Examples 21-24 have good blending uniformity and fluidity, and thus smooth filling of capsules can be achieved.

The capsules prepared in Examples 21-24 were detected for dissolution, and the results show that the dissolution rates at 60 min were all greater than 75%, and thus the capsules can meet the corresponding quality control requirements.

### Comparative Example 1

### Preparation of Oral Capsule Comprising Solid Dispersion Powder of 5-fluoro-1-(4-fluoro-3-(4-(pyrimidin-2-yl)piperazine-1-carbonyl)benzyl)quinazoline-2,4(1H,3H)-dione

The formulation of capsules comprising 10 mg of 5-fluoro-1-(4-fluoro-3-(4-(pyrimidin-2-yl)piperazine-1-carbonyl)benzyl)quinazoline-2,4(1H,3H)-dione per capsule is shown below:

| | Batch A/ | Batch B/ |
|---|---|---|
| | weight | weight |
| Component | (mg) | (mg) |
| Solid dispersion powder of 5-fluoro-1-(4- | 40.00 | 40.00 |
| fluoro-3-(4-(pyrimidin-2-yl)piperazine-1- | | |
| carbonyl)benzyl)quinazoline-2,4(1H,3H)-dione | | |
| Microcrystalline cellulose PH101 | 59.80 | / |
| Microcrystalline cellulose PH102 | / | 59.80 |
| Mannitol 100SD | 119.85 | 119.85 |
| Croscarmellose sodium | 6.90 | 6.90 |
| Colloidal silicon dioxide | 2.30 | 2.30 |
| Magnesium stearate | 1.15 | 1.15 |
| Empty gelatin capsules 0# | 1 capsule | 1 capsule |
| Total amount | 230.00 | 230.00 |

Preparation method for batch A: The solid dispersion powder of 5-fluoro-1-(4-fluoro-3-(4-(pyrimidin-2-yl)piperazine-1-carbonyl)benzyl)quinazoline-2,4(1H,3H)-dione prepared in Example 1, microcrystalline cellulose, mannitol, and croscarmellose sodium were added into a wet granulator and mixed for 5 min at a stirring speed of 400 rpm and a cutting knife speed of 1500 rpm, and then a proper amount of purified water was added for wet granulation. The prepared wet granules were subjected to wet granulation and drying, and the granules obtained after drying and the added colloidal silicon dioxide were mixed at a rotation speed of 15 rpm for 10 min. Magnesium stearate sieved through a 60-mesh sieve was added, and then mixing was performed at a rotation speed of 15 rpm for 3 min. Finally, the obtained mixed granules were filled into empty gelatin capsule shells to obtain oral capsules.

Preparation method for batch B: The solid dispersion powder of 5-fluoro-1-(4-fluoro-3-(4-(pyrimidin-2-yl)piperazine-1-carbonyl)benzyl)quinazoline-2,4(1H,3H)-dione prepared in Example 1, microcrystalline cellulose, mannitol, and croscarmellose sodium were added into a universal mixer and mixed. The obtained mixed material was subjected to dry granulation, and the granules obtained after dry granulation and the added colloidal silicon dioxide were mixed at the lowest rotation speed for 5 min. Magnesium stearate sieved through a 60-mesh sieve was added, and mixing was performed at the lowest rotation speed for 5 min. Finally, the obtained mixed granules were filled into empty gelatin capsule shells to obtain oral capsules.

A wet granulation process was conducted for the batch A, and the prepared mixed granules had good fluidity and stable quality and small weight difference in the capsule filling process. However, the XRPD detection pattern of samples showed that the XRPD pattern of the mixed granules obtained by wet granulation changed compared with the XRPD pattern of the fully blank excipient, and based on the results of the compatibility study between the drug substance and excipients, it may be presumed that the change was mainly caused by the degradation of the hydroxypropyl methylcellulose phthalate in the solid dispersion under the conditions of high temperature and high humidity.

A dry granulation process was conducted for the batch B. During granulation, a large number of materials adhered to the roller, which was mainly caused by the hygroscopicity and a certain cohesiveness of the solid dispersion.

Although the present disclosure has been described in detail, it will be appreciated by those skilled in the art that the same implementations may be performed within a wide and equivalent range of conditions, formulations and other parameters without affecting the scope of the present disclosure or any embodiment thereof. All patents, patent applications, and publications cited herein are incorporated herein by reference in their entirety.

1. A pharmaceutical composition, comprising a solid dispersion powder of an active ingredient 5-fluoro-1-(4-fluoro-3-(4-(pyrimidin-2-yl)piperazine-1-carbonyl)benzyl) quinazoline-2,4(1H,31/)-dione, a filler, a disintegrant, a glidant, and a lubricant, wherein less than 10 wt. %, or less than 5 wt. %, or less than 1 wt. % of the 5-fluoro-1-(4-fluoro-3-(4-(pyrimidin-2-yl)piperazine-1-carbonyl)benzyl)quinazoline-2,4(1H,3H)-dione in the solid dispersion powder is in a crystalline form.

2. The pharmaceutical composition according to claim 1, wherein the solid dispersion powder comprises the active ingredient 5-fluoro-1-(4-fluoro-3-(4-(pyrimidin-2-yl)piperazine-1-carbonyl)benzyl)quinazoline-2,4(1H,31/)-dione, a polymer hydroxypropyl methylcellulose phthalate and optionally a surfactant poloxamer.

3. The pharmaceutical composition according to claim 2, wherein the hydroxypropyl methylcellulose phthalate has a methoxy content of 12.0-28.0%, a 2-hydroxypropoxyl content of 4.0-23.0%, an acetyl content of 2.0-16.0%, and a succinyl content of 4.0-28.0%, calculated on a dried basis.

4. The pharmaceutical composition according to claim 1, wherein,
the filler is selected from a group consisting of starch, sucrose, microcrystalline cellulose, anhydrous calcium hydrophosphate, mannitol, lactose, pregelatinized starch, glucose, maltose, cyclodextrin, cellulose, silicified microcrystalline cellulose and any combination thereof;
the disintegrant is selected from a group consisting of sodium carboxymethyl starch, low-substituted hydroxypropyl cellulose, crospovidone, croscarmellose sodium, croscarmellose, methylcellulose, pregelatinized starch, sodium alginate, and any combination thereof;
the glidant is selected from a group consisting of powdered cellulose, magnesium trisilicate, colloidal silicon dioxide, talc, and any combination thereof;
the lubricant is selected from a group consisting of zinc stearate, glyceryl monostearate, glyceryl palmitostearate, magnesium stearate, sodium stearyl fumarate and any combination thereof; and
the pharmaceutical composition optionally further comprises a binder and/or a solubilizer.

5. The pharmaceutical composition according to claim 1, wherein, based on the total weight of the pharmaceutical composition, the solid dispersion powder has a content of 15-30%, 15-22%, or 16-20%;
the filler has a content of 60-85%, 70-82%, or 75-82%;
the disintegrant has a content of 0.1-10%, or 0.5-3%;
the glidant has a content of 0.1-10%, 0.5-3%, or 1-3%; and
the lubricant has a content of 0.1-3%, 0.3-1%, 0.5±0.2% or 0.5±0.1%.

6. The pharmaceutical composition according to claim 1, wherein, based on the total weight of the pharmaceutical composition, the pharmaceutical composition comprises:
16-20% of the solid dispersion powder, wherein the solid dispersion powder consists of 5-fluoro-1-(4-fluoro-3-(4-(pyrimidin-2-yl)piperazine-1- carbonyl)benzyl)quinazoline-2,4(1H,3H)-dione and hydroxypropyl methylcellulose phthalate in a weight ratio of 1:3, and less than 5 wt. % or less than 1 wt. % of the 5-fluoro-1-(4-fluoro-3-(4-(pyrimidin-2-yl)piperazine-1-carbonyl)benzyl)quinazoline-2,4(1H,3H)-dione is in a crystalline form;
10-28% or 15-28% of microcrystalline cellulose, wherein D90 of the microcrystalline cellulose is 170-480 μm, or 275-480 μm;
50-70%, or 50-65% of mannitol, wherein the particle size distribution of particles with a size of >75 μm of the mannitol is not less than 70%, or not less than 90%;
0.5-3% of crospovidone and/or croscarmellose sodium;
1-3% of colloidal silicon dioxide; and
0.3-1%, 0.5±0.2% or 0.5±0.1%, of magnesium stearate; or
based on the total weight of the pharmaceutical composition, the pharmaceutical composition comprises:
16-20% of a solid dispersion powder, wherein the solid dispersion powder consists of 5-fluoro-1-(4-fluoro-3-(4-(pyrimidin-2-yl)piperazine-1- carbonyl)benzyl)quinazoline-2,4(1H,3H)-dione and hydroxypropyl methylcellulose phthalate in a weight ratio of 1:3, and less than 5 wt. % or less than 1 wt. % of the 5-fluoro-1-(4-fluoro-3-(4-(pyrimidin-2-yl)piperazine-1-carbonyl)benzyl)quinazoline-2,4(1H,3H)-dione is in a crystalline form;
25-55% of microcrystalline cellulose, wherein D90 of the microcrystalline cellulose is 170-480 μm, or 275-480 μm;
25-55% of mannitol, wherein the particle size distribution of particles with a size of >75 μm of the mannitol is not less than 70%, or not less than 90%;
0.5-3% of crospovidone and/or croscarmellose sodium;
1-3% of colloidal silicon dioxide; and
0.3-1%, such as 0.5±0.2% or 0.5±0.1%, of magnesium stearate.

7. A pharmaceutical formulation, wherein the pharmaceutical formulation is a capsule formulation comprising the pharmaceutical composition according to claim 1 and a capsule shell, wherein the capsule shell is selected from a group consisting of a plant capsule shell and a gelatin capsule shell.

8. The pharmaceutical formulation according to claim 7, wherein the capsule formulation is a capsule comprising 10 mg of the active ingredient per capsule, and the pharmaceutical composition in the capsule comprises:
16-20% of a solid dispersion powder, wherein the solid dispersion powder consists of the active ingredient 5-fluoro-1-(4-fluoro-3-(4-(pyrimidin-2-yl)piperazine-1-carbonyl)benzyl)quinazoline-2,4(1H,3H)-dione and hydroxypropyl methylcellulose phthalate in a weight ratio of 1:3, and less than 5 wt. % or less than 1 wt. % of the 5-fluoro-1-(4-fluoro-3-(4-(pyrimidin-2-yl)piperazine-1-carbonyl)benzyl)quinazoline-2,4(1H,3H)-dione is in a crystalline form;
23-28% of microcrystalline cellulose, wherein D90 of the microcrystalline cellulose is 170-480 μm, or 275-480 μm;
50-55% of mannitol, wherein the particle size distribution of particles with a size of >75 μm of the mannitol is not less than 70%, not less than 80%, or not less than 90%;
0.5-3% of crospovidone and/or croscarmellose sodium;
1-3% of colloidal silicon dioxide; and
0.3-1%, or 0.5±0.1%, of magnesium stearate;
or the capsule formulation is a capsule comprising 20 mg of the active ingredient per capsule, and the pharmaceutical composition in the capsule comprises:
16-20% of a solid dispersion powder, wherein the solid dispersion powder consists of the active ingredient 5-fluoro-1-(4-fluoro-3-(4-(pyrimidin-2-yl)piperazine-1-carbonyl)benzyl)quinazoline-2,4(1H,3H)-dione and hydroxypropyl methylcellulose phthalate in a weight ratio of 1:3, and less than 5 wt. % or less than 1 wt. % of the 5-fluoro-1-(4-fluoro-3-(4-(pyrimidin-2-yl)piperazine-1-carbonyl)benzyl)quinazoline-2,4(1H,3H)-dione is in a crystalline form;
12-18% of microcrystalline cellulose, wherein D90 of the microcrystalline cellulose is 170-480 μm or 275-480 μm;
58-63% of mannitol, wherein the particle size distribution of particles with a size of >75 μm of the mannitol is not less than 70%, or not less than 90%;
0.5-3% of crospovidone and/or croscarmellose sodium;
1-3% of colloidal silicon dioxide; and
0.3-1%, or 0.5±0.1%, of magnesium stearate.

9. A method for preparing an oral capsule comprising a solid dispersion powder of 5-fluoro-1-(4-fluoro-3-(4-(pyrimidin-2-yl)piperazine-1-carbonyl)benzyl)quinazoline-2,4(1H,3H)-dione, comprising:
(1) premixing the solid dispersion powder of an active ingredient 5-fluoro-1-(4-fluoro-3-(4-(pyrimidin-2-yl)piperazine-1-carbonyl)benzyl)quinazoline-2,4(1H,3H)-dione, a filler, a disintegrant, and a glidant to obtain a premixture;
(2) sieving the premixture obtained in the step (1) and then mixing again to obtain a first mixture;
(3) sieving a lubricant and adding the lubricant to the first mixture obtained in the step (2), and then mixing to obtain a final mixture; and
(4) filling the final mixture obtained in the step (3) into capsule shells to obtain the oral capsule.

10. The method according to claim 9, wherein the method has one or more of the following features:
the premixing in the step (1) is performed at a rotation speed of 3-40 rpm, 3-20 rpm, or 3-8 rpm for 2-20 min, 2-8 min, or 3-5 min;
the sieving in the step (2) is performed by using a vacuum negative pressure sieve, and a size of a screen used for sieving is 20-40 meshes, or 30 meshes;
the first mixture in the step (2) is obtained by mixing the premixture at a rotation speed of 3-40 rpm, 3-20 rpm, or 3-8 rpm for 3-20 min; 3-15 min, or 6-10 min;
a size of a screen used for sieving in the step (3) is 20-40 meshes, or 30 meshes; and
the mixing in the step (3) is performed at a rotation speed of 3-40 rpm, 3-20 rpm, or 3-8 rpm for 2-20 min; or 6-10 min.

11. The method according to claim 9, wherein the method comprises:
(1) premixing the solid dispersion powder of the active ingredient 5-fluoro-1-(4-fluoro-3-(4-(pyrimidin-2-yl)piperazine-1-carbonyl)benzyl)quinazoline-2,4(1H,3H)-dione, the filler, the disintegrant, and the glidant at a rotation speed of 6 rpm for 3 min to obtain a premixture;
(2) performing manual sieving and mixing the sieved premixture at a rotation speed of 6 rpm for 10 min to obtain a first mixture, wherein a size of a screen used for sieving is 30 meshes;
(3) sieving the lubricant with a 30-mesh sieve, adding the sieved lubricant into the first mixture in the step (2), and mixing at a rotation speed of 6 rpm for 15 min to obtain a final mixture; and
(4) filling the final mixture obtained in the step (3) into capsule shells to obtain the oral capsule;
or the method comprises:
(1) premixing the solid dispersion powder of the active ingredient 5-fluoro-1-(4-fluoro-3-(4-(pyrimidin-2-yl)piperazine-1-carbonyl)benzyl)quinazoline-2,4(1H,3H)-dione, the filler, the disintegrant, and the glidant at a rotation speed of 6 rpm for 3 min to obtain a premixture;
(2) sieving by using a vacuum negative pressure sieve and mixing the sieved premixture at a rotation speed of 6 rpm for 10 min to obtain a first mixture, wherein a size of a screen used for sieving is 30 meshes;
(3) sieving the lubricant with a 30-mesh sieve, adding the sieved lubricant into the first mixture in the step (2), and mixing at a rotation speed of 6 rpm for 3 min to obtain a final mixture; and
(4) filling the final mixture obtained in the step (3) into capsule shells to obtain the oral capsule.

12. The method according to claim 9, wherein the final mixture in the step (4) is a pharmaceutical composition comprising a solid dispersion powder of an active ingredient 5-fluoro-1-(4-fluoro-3-(4-(pyrimidin-2-yl)piperazine-1-carbonyl)benzyl)quinazoline-2,4(1H,3H)-dione, a filler, a disintegrant, a glidant, and a lubricant, wherein less than 10 wt. %, or less than 5 wt. %, or less than 1 wt. % of the 5-fluoro-1-(4-fluoro-3-(4-(pyrimidin-2-yl)piperazine-1- carbonyl)benzyl)quinazoline-2,4(1H,3H)-dione in the solid dispersion powder is in a crystalline form.

13-15. (canceled)

16. The pharmaceutical composition according to claim 2, wherein:
in the solid dispersion powder, the hydroxypropyl methylcellulose phthalate accounts for 65-77% or 73-77% based on a total weight of the solid dispersion powder, the active ingredient accounts for 25-33% based on the total weight of the solid dispersion powder, and optionally, the surfactant accounts for 2-5% based on the total weight of the solid dispersion powder; or
the solid dispersion powder consists of 5-fluoro-1-(4-fluoro-3-(4-(pyrimidin-2-yl)piperazine-1-carbonyl)benzyl)quinazoline-2,4(1H,3H)-dione and the hydroxypropyl methylcellulose phthalate in a weight ratio of 1:2 to 1:3; or
the solid dispersion powder consists of 5-fluoro-1-(4-fluoro-3-(4-(pyrimidin-2-yl)piperazine-1-carbonyl)benzyl)quinazoline-2,4(1H,3H)-dione and the hydroxypropyl methylcellulose phthalate in a weight ratio of 1:2 or 1:3, or consists of 5-fluoro-1-(4-fluoro-3-(4-(pyrimidin-2-yl)piperazine-1-carbonyl)benzyl)quinazoline-2,4(1H,3H)-dione, the hydroxypropyl methylcellulose phthalate and the poloxamer in a weight ratio of 1:2.8:0.2.

17. The pharmaceutical composition according to claim 4, wherein,
the filler is microcrystalline cellulose and/or mannitol; wherein the particle size distribution (D90) of the microcrystalline cellulose is 170-480 μm, 170-283 μm or 275-480 μm; the particle size distribution of particles with a size of >75 μm of the mannitol is not less than 70%, not less than 80%, or not less than 90%;
the disintegrant is selected from a group consisting of crospovidone, croscarmellose, croscarmellose sodium and any combination thereof;
the glidant is colloidal silicon dioxide;
the lubricant is magnesium stearate.

18. The pharmaceutical composition according to claim 5, wherein the filler is microcrystalline cellulose and mannitol, wherein based on the total weight of the pharmaceutical composition, the microcrystalline cellulose has a content of 10-60%, 25-60%, 25-55%, 10-30%, or 15-28%, and the mannitol has a content of 25-70%, 50-70%, 50-68%, 50%-65%, 25-55%, or 25-45%.

19. The method according to claim 9, wherein the oral capsule is a capsule comprising 10 mg of the active ingredient per capsule, and the pharmaceutical composition in the capsule comprises:
16-20% of a solid dispersion powder, wherein the solid dispersion powder consists of the active ingredient 5-fluoro-1-(4-fluoro-3-(4-(pyrimidin-2-yl)piperazine-1-carbonyl)benzyl)quinazoline-2,4(1H,3H)-dione and hydroxypropyl methylcellulose phthalate in a weight ratio of 1:3, and less than 5 wt. % or less than 1 wt. % of the 5-fluoro-1-(4-fluoro-3-(4-(pyrimidin-2-yl)piperazine-1-carbonyl)benzyl)quinazoline-2,4(1H,3H)-dione is in a crystalline form;
23-28% of microcrystalline cellulose, wherein D90 of the microcrystalline cellulose is 170-480 μm or 275-480 μm;
50-55% of mannitol, wherein the particle size distribution of particles with a size of >75 μm of the mannitol is not less than 70%, not less than 80%, or not less than 90%;
0.5-3% of crospovidone and/or croscarmellose sodium;
1-3% of colloidal silicon dioxide; and
0.3-1%, or 0.5±0.1%, of magnesium stearate;
or the oral capsule is a capsule comprising 20 mg of the active ingredient per capsule, and the pharmaceutical composition in the capsule comprises:
16-20% of a solid dispersion powder, wherein the solid dispersion powder consists of the active ingredient 5-fluoro-1-(4-fluoro-3-(4-(pyrimidin-2-yl)piperazine-1-carbonyl)benzyl)quinazoline-2,4(1H,3H)-dione and hydroxypropyl methylcellulose phthalate in a weight ratio of 1:3, and less than 5 wt. % or less than 1 wt. % of the 5-fluoro-1-(4-fluoro-3-(4-(pyrimidin-2-yl)piperazine-1-carbonyl)benzyl)quinazoline-2,4(1H,3H)-dione is in a crystalline form;
12-18% of microcrystalline cellulose, wherein D90 of the microcrystalline cellulose is 170-480 μm or 275-480 μm;
58-63% of mannitol, wherein the particle size distribution of particles with a size of >75 μm of the mannitol is not less than 70% or not less than 90%;
0.5-3% of crospovidone and/or croscarmellose sodium;
1-3% of colloidal silicon dioxide; and
0.3-1%, or 0.5±0.1%, of magnesium stearate.

20. A method for treating or preventing a PARP-mediated disease, comprising administering to a subject in need thereof the pharmaceutical composition according to claim 1 or a capsule formulation comprising the pharmaceutical composition and a capsule shell, wherein the capsule shell is selected from a group consisting of a plant capsule shell and a gelatin capsule shell.

21. The method according to claim 20, wherein the capsule formulation is a capsule comprising 10 mg of the active ingredient per capsule, and the pharmaceutical composition in the capsule comprises:
16-20% of a solid dispersion powder, wherein the solid dispersion powder consists of the active ingredient 5-fluoro-1-(4-fluoro-3-(4-(pyrimidin-2-yl)piperazine-1-carbonyl)benzyl)quinazoline-2,4(1H,3H)-dione and hydroxypropyl methylcellulose phthalate in a weight ratio of 1:3, and less than 5 wt. % or less than 1 wt. % of the 5-fluoro-1-(4-fluoro-3-(4-(pyrimidin-2-yl)piperazine-1-carbonyl)benzyl)quinazoline-2,4(1H,3H)-dione is in a crystalline form;
23-28% of microcrystalline cellulose, wherein D90 of the microcrystalline cellulose is 170-480 μm or 275-480 μm;
50-55% of mannitol, wherein the particle size distribution of particles with a size of >75 μm of the mannitol is not less than 70%, not less than 80%, or not less than 90%;
0.5-3% of crospovidone and/or croscarmellose sodium;
1-3% of colloidal silicon dioxide; and
0.3-1%, or 0.5±0.1%, of magnesium stearate;
or the capsule formulation is a capsule comprising 20 mg of the active ingredient per capsule, and the pharmaceutical composition in the capsule comprises:
16-20% of a solid dispersion powder, wherein the solid dispersion powder consists of the active ingredient 5-fluoro-1-(4-fluoro-3-(4-(pyrimidin-2-yl)piperazine-1-carbonyl)benzyl)quinazoline-2,4(1H,3H)-dione and hydroxypropyl methylcellulose phthalate in a weight ratio of 1:3, and less than 5 wt. % or less than 1 wt. % of the 5-fluoro-1-(4-fluoro-3-(4-(pyrimidin-2-yl)piperazine-1-carbonyl)benzyl)quinazoline-2,4(1H,3H)-dione is in a crystalline form;
12-18% of microcrystalline cellulose, wherein D90 of the microcrystalline cellulose is 170-480 μm or 275-480 μm;
58-63% of mannitol, wherein the particle size distribution of particles with a size of >75 μm of the mannitol is not less than 70% or not less than 90%;
0.5-3% of crospovidone and/or croscarmellose sodium;
1-3% of colloidal silicon dioxide; and
0.3-1%, or 0.5±0.1%, of magnesium stearate.

22. The method according to claim 20, wherein:
the PARP-mediated disease is cancer, and/or
the pharmaceutical formulation further comprises at least one known anti-cancer drug or a pharmaceutically acceptable salt of the anti-cancer drug.

23. The method according to claim 22, wherein:
the cancer is selected from: liver cancer, melanoma, Hodgkin's disease, non-Hodgkin's lymphoma, acute lymphatic leukemia, chronic lymphatic leukemia, multiple myeloma, neuroblastoma, breast cancer, ovarian cancer, lung cancer, Wilms' tumor, cervical cancer, testicular cancer, soft tissue sarcoma, chronic lymphocytic leukemia, primary macroglobulinemia, bladder cancer, chronic myelogenous leukemia, primary brain cancer, malignant melanoma, small cell lung cancer, stomach cancer, colon cancer, malignant pancreatic insulinoma, malignant carcinoid cancer, choriocarcinoma, mycosis fungoides, head and neck cancer, osteoganic sarcoma, pancreatic cancer, acute myeloid leukemia, hairy cell leukemia, rhabdomyosarcoma, Kaposi's sarcoma, genitourinary tumor, thyroid cancer, esophageal cancer, malignant hypercalcemia, cervical hyperplasia, renal cell carcinoma, endometrial cancer, polycythemia vera, essential thrombocythemia, adrenocortical carcinoma, skin cancer and prostate cancer;
the known anti-cancer drug is selected from one or more of the following anti-cancer drugs: busulfan, melphalan, chlorambucil, cyclophosphamide, ifosfamide, temozolomide, bendamustine, cisplatin, mitomycin C, bleomycin, carboplatin, camptothecin, irinotecan, topotecan, doxorubicin, epirubicin, aclacinomycin, mitoxantrone, methylhydroxyellipticine, etoposide, 5-azacytidine, gemcitabine, 5-fluorouracil, methotrexate, 5-fluoro-2′-deoxyuridine, fludarabine, nelarabine, cytarabine, alanosine, pralatrexate, pemetrexed, hydroxyurea, thioguanine, colchicine, vinblastine, vincristine, vinorelbine, paclitaxel, ixabepilone, cabazitaxel, docetaxel, alemtuzumab (Campath), panitumumab, ofatumumab, bevacizumab, herceptin, rituximab, imatinib, gefitinib, erlotinib, lapatinib, sorafenib, sunitinib, nilotinib, dasatinib, pazopanib, temsirolimus, everolimus, vorinostat, romidepsin, tamoxifen, letrozole, fulvestrant, mitoguazone, octreotide, retinoic acid, arsenic trioxide, zoledronic acid, bortezomib, thalidomide or lenalidomide.
